(19) United States
(12) Patent Application Publication
(10) Pub. No.: US 2019/0055576 A1
(43) Pub. Date: Feb. 21, 2019

(54) USE OF HERBICIDE-TOLERANT PROTEIN

(71) Applicants: BEIJING DABEINONG TECHNOLOGY GROUP CO., LTD., Beijing (CN); BEIJING DABEINONG BIOTECHNOLOGY CO., LTD., Beijing (CN)

(72) Inventors: Xiangting XIE, Beijing (CN); Qing TAO, Beijing (CN); Jie PANG, Beijing (CN)

(73) Assignees: BEIJING DABEINONG TECHNOLOGY GROUP CO., LTD., Beijing (CN); BEIJING DABEINONG BIOTECHNOLOGY CO., LTD., Beijing (CN)

(21) Appl. No.: 16/079,252

(22) PCT Filed: Dec. 2, 2016
(86) PCT No.: PCT/CN2016/108410

§ 371 Date: Aug. 23, 2018

(30) Foreign Application Priority Data
Mar. 22, 2016 (CN) 201610164721.5

(51) Int. Cl. C12N 15/82; A01N 43/54; A01N 57/20; C12N 9/16

(52) U.S. Cl. CPC C12N 15/8278; A01N 43/54; C12N 15/8275; C12N 9/16; A01N 57/20

(57) ABSTRACT

The present invention relates to the use of a herbicide-tolerant protein, wherein the method for controlling weeds comprises applying a herbicide containing an effective dose of Sulfometuron-methyl to a plant growth environment where at least one transgenic plant is present, wherein the transgenic plant comprises a nucleotide sequence encoding a thifensulfuron hydrolase in its genome, and compared to other plants without the nucleotide sequence encoding the hydrolase, the transgenic plant has reduced plant damage and/or an increased plant yield. The present invention discloses for the first time that a thifensulfuron hydrolase can show a high tolerance to a Sulfometuron-methyl herbicide, plants containing a nucleotide sequence encoding the thifensulfuron hydrolase are strongly tolerant to the Sulfometuron-methyl herbicide and can at least tolerate 1-fold field concentration, and thus the hydrolase has broad application prospects in plants.

### TECHNICAL FIELD

The present invention relates to the use of a herbicide-tolerant protein, in particular to the use of a thifensulfuron hydrolase to degrade a Sulfometuron-methyl herbicide.

### BACKGROUND ART

Weeds may exhaust valuable nutrients required by crops and other plants of interest in the soil rapidly. Currently, there are many types of herbicides used to control weeds, among which a particularly popular herbicide is glyphosate. Crops resistant to glyphosate have been developed, such as maize, soybean, cotton, sugar beet, wheat, and rice. Therefore, glyphosate can be sprayed onto a field where the glyphosate-resistant crops are planted, so as to control weeds without significant damage to the crops.

Glyphosate has been widely used in the world for more than 20 years, resulting in an over-reliance on glyphosate and glyphosate-tolerant crop technologies, as well as applying a high selection pressure on plants that are naturally more tolerant to glyphosate or have developed a glyphosate-resistant activity in wild weed species. It has been reported that a few weeds have developed resistance to glyphosate, including broad-leaved weeds and gramineous weeds, such as Lolium rigidium, Lohum multiflorum, Eleusine indica Gaertn, Ambrosia artemisiifolia, Conyza canadensis, Conyza bonariensis and Plantago lanceolata. Moreover, weeds that were not agricultural problems before the widespread use of glyphosate-tolerant crops have become prevalent gradually and are difficult to control with glyphosate-tolerant crops, wherein these weeds mainly appear together with (but not only with) broad-leaved weeds that are difficult to control, such as Amaranthus, Chenopodium, dandelion and Commelinaceae species.

In areas where glyphosate-resistant weeds or weed species that are difficult to control are present, growers can compensate for the weakness of glyphosate by tank mixing or switching to other herbicides that may control omitted weeds, such as sulfonylurea herbicides. Sulfonylurea herbicides have become the third herbicide after organophosphorus and acetamide herbicides with global annual sales of not less than $3 billion, and the annual application area of sulfonylurea herbicides in China has been more than 2 million hectares and still shows an expanding trend.

With the emergence of glyphosate-resistant weeds and the expanding application of sulfonylurea herbicides, there is a need to introduce sulfonylurea herbicide tolerance into plants of interest that are sensitive to sulfonylurea herbicides. Sulfonylurea herbicides can be broadly divided into ester bond-containing ones and ester bond-free ones, and there are at least ten remaining types of sulfonylurea herbicides containing ester bonds and having similar chemical structures. It has only been identified that a thifensulfuron hydrolase can degrade thifensulfuron. However, like thifensulfuron, Sulfometuron-methyl also belongs to a sulfonylurea herbicide containing an ester bond, and currently there is no report that thifensulfuron hydrolase is tolerant to a Sulfometuron-methyl herbicide.

### SUMMARY OF THE INVENTION

It is an object of the present invention to provide the use of a herbicide-tolerant protein. Provided for the first time is a method to control field weed growth by applying a herbicide containing an effective dose of Sulfometuron-methyl to a plant growth environment where at least one transgenic plant expressing a thifensulfuron hydrolase is present, increasing the tolerance range of the thifensulfuron hydrolase to the herbicides.

In order to achieve the object above, the present invention provides a method for controlling weeds, comprising applying a herbicide containing an effective dose of Sulfometuron-methyl to a plant growth environment where at least one transgenic plant is present, wherein the transgenic plant comprises a nucleotide sequence encoding a thifensulfuron hydrolase in its genome, and compared to other plants without the nucleotide sequence encoding the thifensulfuron hydrolase, the transgenic plant has reduced plant damage and/or an increased plant yield.

Further, the effective dose of Sulfometuron-methyl is 9-120 g ai/ha.

Furthermore, the transgenic plant is a monocotyledonous plant or a dicotyledonous plant.

Preferably, the transgenic plant is maize, soybean, Arabidopsis thaliana, cotton, rape, rice, sorghum, wheat, barley, millet, sugar cane or oat.

On the basis of the above technical solution, the amino acid sequence of the thifensulfuron hydrolase has an amino acid sequence shown as SEQ ID NO: 1, SEQ ID NO: 4 or SEQ ID NO: 7.

Preferably, the nucleotide sequence of the thifensulfuron hydrolase has:

(a) a nucleotide sequence encoding the amino acid sequence shown as SEQ ID NO: 1, SEQ ID NO: 4 or SEQ ID NO: 7; or

(b) a nucleotide sequence shown as SEQ ID NO: 2 or SEQ ID NO: 3; or

(c) a nucleotide sequence shown as SEQ ID NO: 5 or SEQ ID NO: 6; or

(d) a nucleotide sequence shown as SEQ ID NO: 8 or SEQ ID NO: 9.

Further, the transgenic plant may also comprise at least one second nucleotide different from the nucleotide sequence encoding the thifensulfuron hydrolase.

The second nucleotide encodes a selectable marker protein, a protein with a synthetic activity, a protein with a decomposing activity, an anti-biostress protein, an anti-nonbiostress protein, a male sterile protein, a protein affecting a plant yield and/or a protein affecting plant quality.

Specifically, the second nucleotide encodes 5-enolpyruvylshikimate-3-phosphate synthase, glyphosate oxidoreductase, glyphosate-N-acetyltransferase, glyphosate decarboxylase, glufosinate acetyltransferase, α-ketoglutarate-dependent dioxygenase, dicamba monooxygenase, 4-hydroxyphenylpyruvate dioxygenase, acetolactate synthase, cytochrome-like proteins and/or protoporphyrinogen oxidase.

Optionally, the herbicide containing an effective dose of Sulfometuron-methyl also includes glyphosate herbicides, glufosinate herbicides, auxin herbicides, gramineous herbicides, pre-emergence selective herbicides and/or post-emergence selective herbicides.

In order to achieve the object above, the present invention also provides a method for controlling glyphosate-tolerant weeds, comprising applying an effective dose of a Sulfometuron-methyl herbicide and a glyphosate herbicide to a field where at least one transgenic plant is planted, wherein the field includes glyphosate-tolerant weeds or seeds thereof, the transgenic plant comprises a nucleotide sequence encoding a thifensulfuron hydrolase and a nucleotide sequence encoding a glyphosate-tolerant protein in its genome, and compared to other plants without the nucleotide sequence encoding the thifensulfuron hydrolase and/or the nucleotide sequence encoding the glyphosate-tolerant protein, the transgenic plant has reduced plant damage and/or an increased plant yield.

Further, the effective dose of Sulfometuron-methyl is 9-120 g ai/ha. The effective dose of glyphosate is 200-1600 g ae/ha.

Furthermore, the transgenic plant is a monocotyledonous plant or a dicotyledonous plant.

Preferably, the transgenic plant is maize, soybean, Arabidopsis thaliana, cotton, rape, rice, sorghum, wheat, barley, millet, sugar cane or oat.

On the basis of the above technical solution, the amino acid sequence of the thifensulfuron hydrolase has an amino acid sequence shown as SEQ ID NO: 1, SEQ ID NO: 4 or SEQ ID NO: 7.

Preferably, the nucleotide sequence of the thifensulfuron hydrolase has:

(a) a nucleotide sequence encoding the amino acid sequence shown as SEQ ID NO: 1, SEQ ID NO: 4 or SEQ ID NO: 7; or

(b) a nucleotide sequence shown as SEQ ID NO: 2 or SEQ ID NO: 3; or

(c) a nucleotide sequence shown as SEQ ID NO: 5 or SEQ ID NO: 6; or

(d) a nucleotide sequence shown as SEQ ID NO: 8 or SEQ ID NO: 9.

Further, the glyphosate-tolerant protein includes 5-enolpyruvylshikimate-3-phosphate synthase, glyphosate oxidoreductase, glyphosate-N-acetyltransferase or glyphosate decarboxylase.

Specifically, the amino acid sequence of the glyphosate-tolerant protein has an amino acid sequence shown as SEQ ID NO: 10.

Preferably, the nucleotide sequence of the glyphosate-tolerant protein has:

(a) a nucleotide sequence encoding the amino acid sequence shown as SEQ ID NO: 10; or

(b) a nucleotide sequence shown as SEQ ID NO: 11.

In order to achieve the object above, the present invention also provides a planting system for controlling weed growth, comprising a Sulfometuron-methyl herbicide and a plant growth environment where at least one transgenic plant is present, by applying a herbicide containing an effective dose of Sulfometuron-methyl to the plant growth environment where at least one transgenic plant is present, wherein the transgenic plant comprises a nucleotide sequence encoding a thifensulfuron hydrolase in its genome, and compared to other plants without the nucleotide sequence encoding the thifensulfuron hydrolase, the transgenic plant has reduced plant damage and/or an increased plant yield.

Further, the effective dose of Sulfometuron-methyl is 9-120 g ai/ha.

Furthermore, the transgenic plant is a monocotyledonous plant or a dicotyledonous plant.

Preferably, the transgenic plant is maize, soybean, Arabidopsis thaliana, cotton, rape, rice, sorghum, wheat, barley, millet, sugar cane or oat.

On the basis of the above technical solution, the amino acid sequence of the thifensulfuron hydrolase has an amino acid sequence shown as SEQ ID NO: 1, SEQ ID NO: 4 or SEQ ID NO: 7.

Preferably, the nucleotide sequence of the thifensulfuron hydrolase has:

(a) a nucleotide sequence encoding the amino acid sequence shown as SEQ ID NO: 1, SEQ ID NO: 4 or SEQ ID NO: 7; or

(b) a nucleotide sequence shown as SEQ ID NO: 2 or SEQ ID NO: 3; or

(c) a nucleotide sequence shown as SEQ ID NO: 5 or SEQ ID NO: 6; or

(d) a nucleotide sequence shown as SEQ ID NO: 8 or SEQ ID NO: 9.

Further, the transgenic plant may also comprise at least one second nucleotide different from the nucleotide sequence encoding the thifensulfuron hydrolase.

The second nucleotide encodes a selectable marker protein, a protein with a synthetic activity, a protein with a decomposing activity, an anti-biostress protein, an anti-nonbiostress protein, a male sterile protein, a protein affecting a plant yield and/or a protein affecting plant quality.

Specifically, the second nucleotide encodes 5-enolpyruvylshikimate-3-phosphate synthase, glyphosate oxidoreductase, glyphosate-N-acetyltransferase, glyphosate decarboxylase, glufosinate acetyltransferase, α-ketoglutarate-dependent dioxygenase, 4-hydroxyphenylpyruvate dioxygenase, acetolactate synthase, cytochrome-like proteins and/or protoporphyrinogen oxidase.

Optionally, the herbicide containing a herbicidally effective dose of Sulfometuron-methyl also includes glyphosate herbicides, glufosinate herbicides, auxin herbicides, gramineous herbicides, pre-emergence selective herbicides and/or post-emergence selective herbicides.

In order to achieve the object above, the present invention also provides a planting system for controlling glyphosate-tolerant weeds, comprising a Sulfometuron-methyl herbicide, a glyphosate herbicide and a field where at least one transgenic plant is planted, by applying an effective dose of the Sulfometuron-methyl herbicide and the glyphosate herbicide to the field where at least one transgenic plant is planted, wherein the field includes glyphosate-tolerant weeds or seeds thereof, the transgenic plant comprises a nucleotide sequence encoding a thifensulfuron hydrolase and a nucleotide sequence encoding a glyphosate-tolerant protein in its genome, and compared to other plants without the nucleotide sequence encoding the thifensulfuron hydrolase and/or the nucleotide sequence encoding the glyphosate-tolerant protein, the transgenic plant has reduced plant damage and/or an increased plant yield.

Further, the effective dose of Sulfometuron-methyl is 9-120 g ai/ha. The effective dose of glyphosate is 200-1600 g ae/ha.

Furthermore, the transgenic plant is a monocotyledonous plant or a dicotyledonous plant.

Preferably, the transgenic plant is maize, soybean, Arabidopsis thaliana, cotton, rape, rice, sorghum, wheat, barley, millet, sugar cane or oat.

On the basis of the above technical solution, the amino acid sequence of the thifensulfuron hydrolase has an amino acid sequence shown as SEQ ID NO: 1, SEQ ID NO: 4 or SEQ ID NO: 7.

Preferably, the nucleotide sequence of the thifensulfuron hydrolase has:

(a) a nucleotide sequence encoding the amino acid sequence shown as SEQ ID NO: 1, SEQ ID NO: 4 or SEQ ID NO: 7; or

(b) a nucleotide sequence shown as SEQ ID NO: 2 or SEQ ID NO: 3; or

(c) a nucleotide sequence shown as SEQ ID NO: 5 or SEQ ID NO: 6; or

(d) a nucleotide sequence shown as SEQ ID NO: 8 or SEQ ID NO: 9.

Further, the glyphosate-tolerant protein includes 5-enolpyruvylshikimate-3-phosphate synthase, glyphosate oxidoreductase, glyphosate-N-acetyltransferase or glyphosate decarboxylase.

Specifically, the amino acid sequence of the glyphosate-tolerant protein has an amino acid sequence shown as SEQ ID NO: 10.

Preferably, the nucleotide sequence of the glyphosate-tolerant protein has:

(a) a nucleotide sequence encoding the amino acid sequence shown as SEQ ID NO: 10; or

(b) a nucleotide sequence shown as SEQ ID NO: 11.

In order to achieve the object above, the present invention also provides a method for producing a plant tolerant to a Sulfometuron-methyl herbicide, comprising introducing a nucleotide sequence encoding a thifensulfuron hydrolase into the genome of a plant, wherein when a herbicide containing an effective dose of Sulfometuron-methyl is applied to a field where at least the plant is present, the plant has reduced plant damage and/or an increased plant yield compared to other plants without the nucleotide sequence encoding the thifensulfuron hydrolase.

In order to achieve the object above, the present invention also provides a method for cultivating a plant tolerant to a Sulfometuron-methyl herbicide, comprising:

planting at least one plant propagule, wherein the plant propagule comprises a polynucleotide sequence encoding a thifensulfuron hydrolase in the genome;

growing the plant propagule into a plant;

and applying a herbicide containing an effective dose of Sulfometuron-methyl to a plant growth environment where at least the plant is included and harvesting the plant having reduced plant damage and/or an increased plant yield compared to other plants without the polynucleotide sequence encoding the thifensulfuron hydrolase.

In order to achieve the object above, the present invention also provides a method for protecting a plant from damage caused by a Sulfometuron-methyl herbicide, comprising applying a herbicide containing an effective dose of Sulfometuron-methyl to the plant growth environment where at least one transgenic plant is present, wherein the transgenic plant comprises a nucleotide sequence encoding a thifensulfuron hydrolase in its genome, and compared to other plants without the nucleotide sequence encoding the thifensulfuron hydrolase, the transgenic plant has reduced plant damage and/or an increased plant yield.

In order to achieve the object above, the present invention also provides a method for degrading a Sulfometuron-methyl herbicide with a thifensulfuron hydrolase, comprising applying a herbicide containing an effective dose of Sulfometuron-methyl to the plant growth environment where at least one transgenic plant is present, wherein the transgenic plant comprises a nucleotide sequence encoding the thifensulfuron hydrolase in its genome, and compared to other plants without the nucleotide sequence encoding the thifensulfuron hydrolase, the transgenic plant has reduced plant damage and/or an increased plant yield.

In order to achieve the object above, the present invention also provides the use of a thifensulfuron hydrolase to degrade a Sulfometuron-methyl herbicide.

Specifically, the use of the thifensulfuron hydrolase to degrade a Sulfometuron-methyl herbicide comprises applying a herbicide containing an effective dose of Sulfometuron-methyl to the plant growth environment where at least one transgenic plant is present, wherein the transgenic plant comprises a nucleotide sequence encoding the thifensulfuron hydrolase in its genome, and compared to other plants without the nucleotide sequence encoding the thifensulfuron hydrolase, the transgenic plant has reduced plant damage and/or an increased plant yield.

On the basis of the above technical solution, the amino acid sequence of the thifensulfuron hydrolase has an amino acid sequence shown as SEQ ID NO: 1, SEQ ID NO: 4 or SEQ ID NO: 7.

Preferably, the nucleotide sequence of the thifensulfuron hydrolase has:

(a) a nucleotide sequence encoding the amino acid sequence shown as SEQ ID NO: 1, SEQ ID NO: 4 or SEQ ID NO: 7; or

(b) a nucleotide sequence shown as SEQ ID NO: 2 or SEQ ID NO: 3; or

(c) a nucleotide sequence shown as SEQ ID NO: 5 or SEQ ID NO: 6; or

(d) a nucleotide sequence shown as SEQ ID NO: 8 or SEQ ID NO: 9.

The transgenic plant in the present invention is planted in the soil of the plant growth environment within 21 days after applying the herbicide. Optionally, the herbicide can be applied before, simultaneously with or after planting the transgenic plant. Specifically, the transgenic plant is planted in the soil 12, 10, 7 or 3 days before applying the herbicide; or the transgenic plant is planted in the soil 12, 10, 7 or 3 days after applying the herbicide. A second treatment can be further performed on the transgenic plant with the herbicide, wherein the second treatment may be between the V1-V2 stage and the V3-V4 stage, before flowering, at the flowering time, after flowering or at the seeding time.

The sulfometuron-methyl in the present invention refers to 2-(4,6-dimethyl pyrimidine-2-amino-formamido-sulfonyl) methyl benzoate 2-(4,6-dimethyl pyrimidine-2-amino-formamido-sulfonyl) methyl benzoate. Common reagents are 10% sulfometuron-methyl wettable powder, 10% sulfometuron-methyl water suspension (also called dry suspension agent or dry flowable). Commercial reagents of sulfometuron-methyl include but are not limited to oust and sulfometuron-methyl.

The effective dose of sulfometuron-methyl in the present invention refers to a usage amount of 9-120 g ai/ha, including 10-100 g ai/ha, 15-90 g ai/ha, 20-80 g ai/ha, 25-70 g ai/ha, 30-60 g ai/ha or 40-50 g ai/ha.

The dicotyledonous plant in the present invention includes, but is not limited to, alfalfa, bean, cauliflower, cabbage, carrot, celery, cotton, cucumber, eggplant, lettuce, melon, pea, pepper, zucchini, radish, rape, spinach, soybean, pumpkin, tomato, Arabidopsis thaliana or watermelon. Preferably, the dicotyledonous plant refers to soybean, Arabidopsis thaliana, cotton or rape.

The monocotyledonous plant in the present invention includes, but is not limited to, maize, rice, sorghum, wheat, barley, rye, millet, sugar cane, oat or turfgrass. Preferably, the monocotyledonous plant refers to maize, rice, sorghum, wheat, barley, millet, sugar cane or oat.

In the present invention, the herbicide-tolerant protein is a thifensulfuron hydrolase, such as shown as SEQ ID NO: 1, SEQ ID NO: 4 and SEQ ID NO: 7 in the sequence listing. The herbicide-tolerant gene is a nucleotide sequence encoding the thifensulfuron hydrolase, such as shown as SEQ ID NO: 2, SEQ ID NO: 3, SEQ ID NO: 5, SEQ ID NO: 6, SEQ ID NO: 8 and SEQ ID NO: 9 in the sequence listing. For use in a plant, in addition to the coding region for the thifensulfuron hydrolase, the herbicide-tolerant gene may comprise other elements, e.g., ones encoding a selectable marker protein, a protein with a synthetic activity, a protein with a decomposing activity, an anti-biostress protein, an anti-nonbiostress protein, a male sterile protein, a protein affecting plant yield and/or a protein affecting plant quality, thus obtaining a transgenic plant having a herbicide-tolerant activity and other traits.

The anti-biostress protein in the present invention refers to a protein resistant to stresses imposed by other organisms, such as an insect-resistant protein and a (virus, bacterium, fungus and nematode) disease-resistant protein.

The anti-nonbiostress protein in the present invention refers to a protein resistant to stresses imposed by the external environment, such as proteins tolerant to a herbicide, drought, heat, cold, freezing, salt stress, oxidative stress, etc.

The protein affecting plant quality in the present invention refers to a protein affecting a plant output trait, such as a protein improving the quality and content of starch, oil, vitamins and the like, and a protein improving fiber quality.

In addition, an expression cassette comprising the nucleotide sequence encoding the thifensulfuron hydrolase may further be expressed together with at least one protein encoding a herbicide-tolerant gene in a plant, wherein the herbicide-tolerant gene includes, but is not limited to, 5-enolpyruvylshikimate-3-phosphate synthase (EPSPS), glyphosate oxidoreductase (GOX), glyphosate-N-acetyltransferase (GAT), glyphosate decarboxylase, glufosinate acetyltransferase (PAT), α-ketoglutarate-dependent dioxygenase (AAD), dicamba monooxygenase (DMO), 4-hydroxyphenylpyruvate dioxygenase (HPPD), acetolactate synthase (ALS), cytochrome-like proteins (P450) and/or protoporphyrinogen oxidase (Protox).

The “glyphosate” in the present invention refers to N-phosphonomethylglycine and salts thereof. Treating with a “glyphosate herbicide” refers to performing treatment using any glyphosate-containing herbicide preparation. Commercial preparations of glyphosate include, but are not limited to, ROUNDUP® (as an isopropylamine salt of glyphosate), ROUNDUP® WEATHERMAX (as a potassium salt of glyphosate), ROUNDUP® DRY and RIVAL® (as an amine salt of glyphosate), ROUNDUP® GEOFORCE (as a sodium salt of glyphosate) and TOUCHDOWN® (as a trimethylsulfonium salt of glyphosate).

The effective dose of glyphosate in the present invention refers to a usage amount of 200-1600 g ae/ha, including 250-1600 g ae/ha, 300-1600 g ae/ha, 500-1600 g ae/ha, 800-1500 g ae/ha, 1000-1500 g ae/ha or 1200-1500 g ae/ha.

The “glufosinate” (also known as phosphinothricin) in the present invention refers to ammonium 2-amino-4-[hydroxy(methyl)phosphonyl]butyrate. Treating with a “glufosinate herbicide” refers to performing treatment using any glufosinate-containing herbicide preparation.

The auxin herbicides in the present invention simulate natural plant growth regulators called auxin or act as the regulators, wherein the herbicides affect cell wall plasticity and nucleic acid metabolism, resulting in uncontrolled cell division and growth. Damage symptoms caused by the auxin herbicides include epinastic bending or twisting of stems and petioles, cup-shaped or curled leaves and abnormal leaf shapes and veins. The auxin herbicides include, but are not limited to, phenoxycarboxylic acid compounds, benzoic acid compounds, pyridinecarboxylic acid compounds, quinolinecarboxylic acid compounds or benazolin-ethyl compounds. Typically, the auxin herbicides are dicamba, 2,4-dichlorophenoxy acetic acid (2,4-D), (4-chloro-2-methylphenoxy)acetic acid (MCPA) and/or 4-(2,4-dichlorophenoxy)butyric acid (2,4-DB).

The “dicamba” in the present invention refers to 3,6-dichloro-o-anisic acid or 3,6-dichloro-2-methoxybenzoic acid and acids and salts thereof, in which the salts thereof include isopropylamine salt, diglycolamine salt, dimethylamine salt, potassium salt and sodium salt. Commercial preparations of dicamba include, but are not limited to, Banvel® (as a DMA salt), Clarity® (BASF, as a DGA salt), VEL-58-CS-11™ and Vanquish® (BASF, as a DGA salt).

The gramineous herbicides in the present invention are not used in maize unless maize is already tolerant thereto, and such tolerance may be provided via α-ketoglutarate-dependent dioxygenase (e.g., the AAD gene), wherein the gramineous herbicides include, but are not limited to, fluazifop-p-butyl.

The pre-emergence selective herbicides in the present invention include, but are not limited to, acetanilide, acetochlor, acetolactate synthase inhibitors, dinitroaniline or protoporphyrinogen oxidase inhibitors.

The post-emergence selective herbicides in the present invention include, but are not limited to, nicosulfuron, rimsulfuron, 2,4-D, dicamba, fluoroglycofen-ethyl and quizalofop-p-ethyl.

The application amount of the herbicide in the present invention varies depending on the soil structure, the pH value, the organic content, the tillage system and the weed size, and is determined by viewing the suitable herbicide application amount on a herbicide label.

Weeds that can be controlled by the sulfometuron-methyl herbicide in the present invention include, but are not limited to annual and perennial grasses and broad leaved grasses, such as Johnson grass, silk-leaved eupatorium, sheep fescue, willow herb, solidago decurre, fleabane, blue grass, chufa, monarch, ragweed, revered-leaved eupatorium, yellow sweet clover, etc.

Weeds that can be controlled by the glyphosate herbicide in the present invention include, but are not limited to, Alopecurus myosuroides, Avena fatua, Bromus japonicus, Aponogeton madagascarirnsi, Echinochloa crus-galli, Poa annua, Setaria viridis, Digitaria sanguinalis, Portulaca oleracea, Chenopodium album, Xanthium strumarium, Abutilon theophrasti, Polygonum, Plantago asiatica, Stellaria media, Galium aparine, sedges, etc.

The planting system in the present invention refers to a combination of a plant and any herbicide tolerance shown thereby and/or an available herbicide treatment in different plant developmental stages, producing a high-yielding and/or damage-reduced plant.

Glyphosate is widely used, as it controls a very broad spectrum of broad-leaved and gramineous weed species. However, reusing glyphosate in glyphosate-tolerant crops and non-crop applications has been (and still continues to be) chosen to make weeds evolve into naturally more tolerant species or glyphosate-resistant biotypes. Most herbicide resistance management strategies suggest using an effective amount of various herbicides as a means of delaying the emergence of resistant weeds, wherein the various herbicides provide control of the same species, but have different modes of action. Superposing the thifensulfuron hydrolase gene with a glyphosate tolerance trait (and/or another herbicide tolerance trait) can achieve control of glyphosate-resistant weed species (broad-leaved weed species controlled by the Sulfometuron-methyl herbicide) in glyphosate-tolerant crops by allowing for selective use of glyphosate and Sulfometuron-methyl on the same crop. The application of these herbicides can be performed by using simultaneously in a tank mixture containing two or more herbicides with different modes of action, or using a single herbicide composition alone in continuous use (e.g., before planting, before or after emergence) (with an interval time range used being from 2 hours to 3 months), or alternatively, can be performed by using a combination of any number of herbicides representative of each applicable compound category at any time (from 7 months after planting a crop to the time when the crop is harvested (or the pre-harvest interval for a single herbicide, taking the shortest)).

A herbicide preparation (e.g., an ester, acid or salt-formulated or soluble concentrate, emulsifying concentrate or soluble liquid) and a tank mix additive (e.g., an adjuvant or compatilizer) can significantly affect weed control of a given herbicide or a combination of one or more herbicides. Any chemical combination of any of the foregoing herbicides is within the scope of the present invention.

In the present invention, weeds refer to plants competing with the cultivated plants in the plant growth environment.

The term “control” and/or “prevention” in the present invention refers to at least direct application of (e.g., by spraying) an effective dose of a Sulfometuron-methyl herbicide to the plant growth environment, so as to minimize weed development and/or stop weeds from growing. At the same time, the cultivated plants should be morphologically normal and can be cultivated under conventional methods for product consumption and/or production; and preferably, compared to non-transgenic wild-type plants, the cultivated plants have reduced plant damage and/or an increased plant yield. Specific performances of reduced plant damage include, but are not limited to, an improved stem resistance and/or an increased grain weight, etc. The “control” and/or “prevention” effect of the thifensulfuron hydrolase on weeds can exist independently, and will not be diminished and/or lost due to the presence of other substances that can “control” and/or “prevent” the weeds. Specifically, if any tissue of a transgenic plant (containing the polynucleotide sequence encoding the thifensulfuron hydrolase) has and/or produces the thifensulfuron hydrolase and/or another substance that can control weeds simultaneously and/or asynchronously, then the presence of the another substance will neither affect the “control” and/or “prevention” effect of the thifensulfuron hydrolase on the weeds, nor result in the “control” and/or “prevention” effect being completely and/or partially achieved by the another substance regardless of the thifensulfuron hydrolase.

In the present invention, expression of the thifensulfuron hydrolase in a transgenic plant can be accompanied by the expression of one or more other herbicide-tolerant proteins. This co-expression of more than one herbicide-tolerant protein in the same transgenic plant can be achieved by allowing the plant to comprise and express a desired gene through genetic engineering. In addition, a plant (a first parent) can express the thifensulfuron hydrolase through genetic engineering manipulation, and a second plant (a second parent) can express other herbicide-tolerant proteins through genetic engineering manipulation. Progeny plants expressing all the genes introduced into the first parent and the second parent are obtained by hybridizing the first parent with the second parent.

The genome of a plant, plant tissue or plant cell in the present invention refers to any genetic material within the plant, plant tissue or plant cell, and includes nuclear, plastid and mitochondrial genomes.

The “plant propagule” in the present invention includes, but is not limited to, plant sexual propagules and plant vegetative propagules. The plant sexual propagules include, but are not limited to, plant seeds; and the plant vegetative propagules refer to vegetative organs or a specific tissue of a plant, which can generate a new plant under ex vivo conditions, wherein the vegetative organs or the specific tissue include, but are not limited to, roots, stems and leaves, for example: plants with roots as the vegetative propagules include strawberry, sweet potato and the like; plants with stems as the vegetative propagules include sugar cane, potato (tuber) and the like; and plants with leaves as the vegetative propagules include aloe, begonia and the like.

The “resistance” in the present invention is heritable, and allows a plant to grow and propagate in the case where an effective treatment by a general herbicide is performed on a given plant. As recognized by a person skilled in the art, even if a certain degree of damage of a plant treated with a herbicide is apparent, the plant can still be considered “resistant”. The term “tolerant” or “tolerance” in the present invention is more extensive than the term “resistance”, and includes “resistance” and an improved ability of a particular plant to resist various degrees of damage induced by a herbicide, and generally damage to a wild-type plant with the same genotype can be caused at the same herbicide dose.

The polynucleotide and/or nucleotide in the present invention forms a complete “gene”, which encodes a protein or a polypeptide in a desired host cell. A person skilled in the art will readily appreciate that the polynucleotide and/or nucleotide in the present invention can be placed under the control of a regulatory sequence in a host of interest.

As well known to a person skilled in the art, DNA is typically present in a double-stranded form. In this arrangement, one strand is complementary to the other, and vice versa. The other complementary strand of DNA is produced since DNA is replicated in a plant. As such, the present invention includes the use of the polynucleotides and complementary strands thereof exemplified in the sequence listing. “Coding strand” commonly used in the art refers to a strand bound to an anti-sense strand. In order to express a protein in vivo, one strand of DNA is typically transcribed to one mRNA complementary strand, which acts as a template to translate the protein. Actually, mRNA is transcribed from the “anti-sense” strand of DNA. The “sense” or “coding” strand has a series of codons (a codon is composed of three nucleotides, and a specific amino acid can be produced by reading three codons at a time), which can be read as an open reading frame (ORF) to form a protein or peptide of interest. The present invention also includes RNA with an equivalent function to the exemplary DNA.

The nucleic acid molecule or a fragment thereof in the present invention hybridizes with the herbicide-tolerant gene of the present invention under stringent conditions. Any conventional nucleic acid hybridization or amplification method can be used to identify the presence of the herbicide-tolerant gene of the present invention. A nucleic acid molecule or a fragment thereof is capable of specifically hybridizing with other nucleic acid molecules under certain circumstances. In the present invention, if two nucleic acid molecules can form an anti-parallel double-stranded nucleic acid structure, then it can be considered that these two nucleic acid molecules can be specifically hybridized with each other. If two nucleic acid molecules exhibit a complete complementarity, then one nucleic acid molecule of the two is said to be the “complement” of the other nucleic acid molecule. In the present invention, when each nucleotide of a nucleic acid molecule is complementary to the corresponding nucleotide of another nucleic acid molecule, then these two nucleic acid molecules are said to exhibit a “complete complementarity”. If two nucleic acid molecules can be hybridized with each other with a sufficient stability to allow them to anneal and bind with each other at least under conventional “low stringency” conditions, then these two nucleic acid molecules are said to be “minimally complementary”. Similarly, if two nucleic acid molecules can be hybridized with each other with a sufficient stability to allow them to anneal and bind with each other under conventional “high stringency” conditions, then these two nucleic acid molecules are said to be “complementary”. Deviation from a complete complementarity is permissible, as long as this deviation does not completely prevent two molecules from forming a double-stranded structure. In order to enable a nucleic acid molecule to act as a primer or probe, it is only guaranteed that the molecule has a sufficient complementarity in its sequence to allow a stable double-stranded structure to be formed at the particular solvent and salt concentration employed.

In the present invention, a substantially homologous sequence is a segment of a nucleic acid molecule, wherein the nucleic acid molecule can be specifically hybridized with the complementary strand of another segment of a matched nucleic acid molecule under high stringency conditions. Suitable stringent conditions that promote DNA hybridization are for example treating with 6.0× sodium chloride/sodium citrate (SSC) under the condition of approximately 45° C., and then washing with 2.0×SSC under the condition of 50° C., which conditions are well known to a person skilled in the art. For example, the salt concentration in the washing step can be selected from the low stringency condition of about 2.0×SSC, 50° C. to the high stringency condition of about 0.2×SSC, 50° C. In addition, the temperature condition in the washing step can rise from the low stringency condition of room temperature (about 22° C.) to the high stringency condition of about 65° C. The temperature condition and the salt concentration can both vary, and it is also possible that one of the two remains unchanged while the other variable varies. Preferably, the stringent conditions in the present invention can be specifically hybridizing a sequence with the nucleotide sequence of the thifensulfuron hydrolase in the present invention in a 6×SSC, 0.5% SDS solution at 65° C., and then washing the membrane once with 2×SSC, 0.1% SDS and 1×SSC, 0.1% SDS respectively.

Consequently, sequences which have herbicide tolerance activity and are hybridized with the nucleotide sequence of the thifensulfuron hydrolase in the present invention under stringent conditions are included in the present invention. These sequences are at least approximately 40%-50% homologous, approximately 60%, 65% or 70% homologous to the sequence of the present invention, and even have a sequence homology of at least approximately 75%, 80%, 85%, 90%, 91%, 92%, 93%, 94%, 95%, 96%, 97%, 98%, 99% or more with the sequence of the present invention.

The present invention provides a functional protein. In the present invention, “functional activity” (or “activity”) means that the protein/enzyme used in the present invention (alone or in combination with other proteins) has the ability to degrade a herbicide or diminish the herbicide activity. A plant producing the protein of the present invention preferably produces an “effective amount” of the protein, so that when treating the plant with a herbicide, the protein expression level is sufficient to confer the plant a complete or partial resistance or tolerance to the herbicide (unless otherwise specified, a general amount). The herbicide can be used in an amount usually killing a target plant or a normal field amount and concentration. Preferably, the plant cell and plant of the present invention are protected from growth inhibition or damage caused by treatment with the herbicide. The transformed plant and plant cell of the present invention are preferably tolerant or resistant to a Sulfometuron-methyl herbicide, that is, the transformed plant and plant cell are able to grow in the presence of an effective amount of the Sulfometuron-methyl herbicide.

The gene and protein in the present invention not only comprise a specific exemplary sequence, but also comprise a portion and/or a fragment (including an internal deletion and/or terminal deletion compared to the full-length protein), a variant, a mutant, a substitute (a protein having substituted amino acids), a chimera and a fusion protein which retain the herbicide tolerance activity characteristic of the specific exemplary protein. The “variant” or “variation” refers to a nucleotide sequence that encodes the same protein or encodes an equivalent protein having a herbicide resistance activity. The “equivalent protein” refers to a protein having the same or substantially the same herbicide tolerance bioactivity as the claimed protein.

The “fragment” or “truncation” of a DNA molecule or protein sequence in the present invention refers to a portion of the original DNA or protein sequence (nucleotides or amino acids) involved or an artificially modified form thereof (e.g., a sequence suitable for plant expression), wherein the length of the foregoing sequences may vary, but the length is sufficient to ensure that the (encoded) protein is a herbicide-tolerant protein.

Owing to the degeneracy of the genetic codon, a variety of different DNA sequences may encode the same amino acid sequence. It is within the skill of a person skilled in the art to produce these alternative DNA sequences encoding the same or substantially the same protein. These different DNA sequences are included in the scope of the present invention. A “substantially the same” sequence refers to a sequence with an amino acid substitution, deletion, addition or insertion that does not substantively affect the herbicide tolerance activity, wherein a fragment retaining the herbicide tolerance activity is also included.

The substitution, deletion or addition of an amino acid sequence in the present invention is a conventional technique in the art, and preferably, this amino acid change is: a small characteristic change, that is a conservative amino acid substitution that does not significantly affect the folding and/or activity of a protein; a small deletion, typically a deletion of about 1-30 amino acids; a small amino or carboxyl terminal extension, e.g., a methionine residue extending at the amino terminus; or a small linker peptide, e.g., about 20-25 residues in length.

Examples of conservative substitutions are substitutions occurring within the following amino acid groups: basic amino acids (e.g., arginine, lysine and histidine), acidic amino acids (e.g., glutamic acid and aspartic acid), polar amino acids (e.g., glutamine and asparagine), hydrophobic amino acids (e.g., leucine, isoleucine and valine), aromatic amino acids (e.g., phenylalanine, tryptophan and tyrosine) and small molecule amino acids (e.g., glycine, alanine, serine, threonine and methionine). Those amino acid substitutions that generally do not alter the specific activity are well known in the art, and have been described, for example, by N. Neurath and R. L. Hill in Protein published by Academic Press, New York, 1979. The most common substitutions are Ala/Ser, Val/Ile, Asp/Glu, Thu/Ser, Ala/Thr, Ser/Asn, Ala/Val, Ser/Gly, Tyr/Phe, Ala/Pro, Lys/Arg, Asp/Asn, Leu/Ile, Leu/Val, Ala/Glu and Asp/Gly, as well as reverse substitutions thereof.

As will be apparent to a person skilled in the art, this substitution can occur outside the region that is important for molecular function, and still produces an active polypeptide. Amino acid residues that are essential for the activity of the polypeptide of the present invention and thus are chosen not to be substituted can be identified according to methods known in the art, such as site-directed mutagenesis or alanine-scanning mutagenesis (see for reference, Cunningham and Wells, 1989, Science 244: 1081-1085). The latter technique is to introduce a mutation to each positively charged residue in a molecule and detect the herbicide resistance activity of the resulting mutant molecule to determine amino acid residues that are important for the molecular activity. Substrate-enzyme interaction sites can also be determined by analyzing the three-dimensional structure thereof, wherein this three-dimensional structure can be determined by nuclear magnetic resonance analysis, crystallography, photoaffinity labelling and other techniques (see, e.g., de Vos et al., 1992, Science 255: 306-312; Smith et al., 1992, J. Mol. Biol 224:899-904; and Wlodaver et al., 1992, FEBS Letters 309: 59-64).

In the present invention, the amino acid sequence encoding the thifensulfuron hydrolase includes, but is not limited to, the sequences involved in the sequence listing of the present invention, and amino acid sequences with a certain degree of homology thereto are also included in the present invention. The similarity/identity of these sequences to the sequence of the present invention is typically greater than 60%, preferably greater than 75%, more preferably greater than 80%, even more preferably greater than 90%, and may be greater than 95%. Preferred polynucleotides and proteins of the present invention can also be defined according to a more specific range of identity and/or similarity. For example, these sequences have an identity and/or similarity of 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% to the exemplary sequence of the present invention.

The regulatory sequence in the present invention includes, but is not limited to, a promoter, a transit peptide, a terminator, an enhancer, a leader sequence, an intron and other regulatory sequences operably linked to the thifensulfuron hydrolase gene.

The promoter is a plant expressible promoter. The “plant expressible promoter” refers to a promoter that ensures the expression of the coding sequence linked thereto in a plant cell. The plant expressible promoter can be a constitutive promoter. Examples of the promoters directing the constitutive expression in plants include, but are not limited to, 35S promoter derived from cauliflower mosaic virus, maize Ubi promoters, rice GOS2 gene promoters, and the like. Alternatively, the plant expressible promoter can be a tissue specific promoter, i.e. the promoter directs the expression of a coding sequence in several tissues such as green tissues at a level higher than in other tissues of the plant (which can be measured through conventional RNA trials), such as a PEP carboxylase promoter. Alternatively, the plant expressible promoter can be a wound-inducible promoter. The wound-inducible promoter or a promoter directing a wound-induced expression pattern means that when a plant suffers from wound caused by a mechanical factor or gnawing of insects, the expression of the coding sequence under the regulation of the promoter is significantly improved compared with under normal growth conditions. Examples of the wound-inducible promoters include, but are not limited to, promoters of potato and tomato protease inhibitor genes (pinI and pinII) and maize protease inhibitor gene (MPI).

The transit peptide (also known as secretion signal sequence or targeting sequence) directs a transgenic product to a specific organelle or cell compartment. For a receptor protein, the transit peptide may be heterologous, for example, targeting the chloroplast using a sequence encoding the chloroplast transit peptide, or targeting the endoplasmic reticulum using a ‘KDEL’ retention sequence, or targeting the vacuole using CTPP of the barley phytolectin gene.

The leader sequence includes, but is not limited to, a small RNA virus leader sequence, such as EMCV leader sequence (5′ non-coding region of encephlomyocarditis virus); a potato virus Y group leader sequence, such as MDMV (Maize Dwarf Mosaic Virus) leader sequence; human immunoglobulin heavy chain binding protein (BiP); an untranslated leader sequence of the coat protein mRNA of alfalfa mosaic virus (AMV RNA4); and a tobacco mosaic virus (TMV) leader sequence.

The enhancer includes, but is not limited to, cauliflower mosaic virus (CaMV) enhancer, figwort mosaic virus (FMV) enhancer, carnation etched ring virus (CERV) enhancer, cassava vein mosaic virus (CsVMV) enhancer, mirabilis mosaic virus (MMV) enhancer, cestrum yellow leaf curling virus (CmYLCV) enhancer, cotton leaf curl Multan virus (CLCuMV) enhancer, commelina yellow mottle virus (CoYMV) enhancer and peanut chlorotic streak caulimovirus (PCLSV) enhancer.

For use in a monocotyledonous plant, the intron includes, but is not limited to, maize hsp70 intron, maize ubiquitin intron, Adh intron 1, sucrose synthase intron or rice Act1 intron. For use in a dicotyledonous plant, the intron includes, but is not limited to, CAT-1 intron, pKANNIBAL intron, PIV2 intron and “super ubiquitin” intron.

The terminator can be a suitable polyadenylation signal sequence that functions in a plant, including, but not limited to, a polyadenylation signal sequence derived from the Agrobacterium tumefaciens nopaline synthetase (NOS) gene, a polyadenylation signal sequence derived from the protease inhibitor II (pinII) gene, a polyadenylation signal sequence derived from the pea ssRUBISCO E9 gene and a polyadenylation signal sequence derived from the α-tubulin gene.

The “effectively linking” in the present invention indicates binding of a nucleic acid sequence, wherein the binding enables a sequence to provide a function required for the linked sequence. The “effectively linking” in the present invention can link a promoter to a sequence of interest, so that the transcription of the sequence of interest is controlled and regulated by the promoter. When a sequence of interest encodes a protein and the expression of the protein is desired, “effectively linking” means that: a promoter is linked to the sequence in such a manner that the resulting transcript is efficiently translated. If the linking of a promoter to a coding sequence is transcript fusion and expression of the encoded protein is intended to be achieved, such linking is created that the first translation initiation codon in the resulting transcript is the initiation codon in the coding sequence. Alternatively, if the linking of a promoter to a coding sequence is translation fusion and expression of the encoded protein is intended to be achieved, such a linking is created that the first translation initiation codon contained in the 5′ untranslated sequence is linked to the promoter in such a manner that the relationship of the resulting translation product with the translation open reading frame encoding the desired protein is in-frame. Nucleic acid sequences that can be “effectively linked” include, but are not limited to: sequences providing gene expression functions (i.e., gene expression elements, such as promoters, 5′ untranslated regions, introns, protein coding regions, 3′ untranslated regions, polyadenylation sites and/or transcription terminators), sequences providing DNA transfer and/or integration functions (i.e., T-DNA boundary sequences, site-specific recombinase recognition sites and integrase recognition sites), sequences providing selective functions (i.e., antibiotic resistance markers and biosynthesis genes), sequences providing marker scoring functions, sequences assisting in sequence manipulation in vitro or in vivo (i.e., polylinker sequences and site-specific recombination sequences) and sequences providing replication functions (i.e., bacterial origins of replication, autonomously replicating sequences and centromeric sequences).

The present invention may confer a new herbicide resistance trait to a plant, and no adverse effects on phenotypes, including yields, are observed. The plant in the present invention can tolerate, for example, 2×, 3×, 4× or 5× the general application level of at least one herbicide tested. The improvement of these levels of tolerance is within the scope of the present invention. For example, foreseeable optimization and further development can be performed on various techniques known in the art, to increase the expression of a given gene.

The thifensulfuron hydrolase in the present invention is tolerant to a Sulfometuron-methyl herbicide. The plant in the present invention contains an exogenous DNA in its genome, wherein the exogenous DNA comprises a nucleotide sequence encoding the thifensulfuron hydrolase, and the plant is protected from the threat of a herbicide by expressing an effective amount of the protein. The effective amount refers to a dose causing no or minor damage. At the same time, the plant should be morphologically normal and can be cultivated under conventional methods for product consumption and/or production.

The expression level of the herbicide-tolerant protein in a plant material can be detected by a variety of methods described in the art, for example, quantifying the mRNA encoding the herbicide-tolerant protein produced in a tissue by applying specific primers, or specifically detecting the amount of herbicide-tolerant protein produced directly.

In the present invention, an exogenous DNA is introduced into a plant, for example introducing a gene or expression cassette or recombinant vector encoding the thifensulfuron hydrolase into a plant cell. Conventional transformation methods include, but are not limited to, Agrobacterium-mediated transformation, microprojectile bombardment, directly uptaking DNA into the protoplast, electroporation or silicon whisker-mediated DNA introduction.

The present invention provides the use of a herbicide-tolerant protein, having the following advantages:

1. Having a broad herbicide tolerance. The present invention discloses for the first time that a thifensulfuron hydrolase can show a high tolerance to a Sulfometuron-methyl herbicide, thus having broad application prospects in plants.

2. Having a strong herbicide tolerance. The thifensulfuron hydrolase of the present invention is strongly tolerant to a Sulfometuron-methyl herbicide and can at least tolerate 1-fold field concentration.

The technical solution of the present invention is further described in detail through drawings and examples below.

### DESCRIPTION OF THE DRAWINGS

FIG. 1 is a construction flow chart of a recombinant cloning vector DBN01-T containing an ALT nucleotide sequence for the use of the herbicide-tolerant protein of the present invention;

FIG. 2 is a construction flow chart of a recombinant expression vector DBN100632 containing an ALT nucleotide sequence for the use of the herbicide-tolerant protein of the present invention;

FIG. 3 is a schematic structural diagram of a recombinant expression vector DBN100631 containing an ALT nucleotide sequence for the use of the herbicide-tolerant protein of the present invention;

FIG. 4 is an effect diagram of the tolerance of a transgenic Arabidopsis thaliana T₁ plant to a Sulfometuron-methyl herbicide for the use of the herbicide-tolerant protein of the present invention;

FIG. 5 is a construction flow chart of a recombinant expression vector DBN100828 containing an ALT nucleotide sequence for the use of the herbicide-tolerant protein of the present invention;

FIG. 6 is a schematic structural diagram of a recombinant expression vector DBN100827 containing an ALT nucleotide sequence for the use of the herbicide-tolerant protein of the present invention;

FIG. 7 is a construction flow chart of a recombinant cloning vector DBN05-T containing an ALT nucleotide sequence for the use of the herbicide-tolerant protein of the present invention;

FIG. 8 is a construction flow chart of a recombinant expression vector DBN100830 containing an ALT nucleotide sequence for the use of the herbicide-tolerant protein of the present invention;

FIG. 9 is a schematic structural diagram of a recombinant expression vector DBN100829 containing an ALT nucleotide sequence for the use of the herbicide-tolerant protein of the present invention.

### PARTICULAR EMBODIMENTS

The technical solution of the use of the herbicide-tolerant protein of the present invention is further described through specific examples below.

### Example 1. Acquisition and Synthesis of an ALT Gene Sequence

1. Acquisition of an ALT Gene Sequence

The amino acid sequence (398 amino acids) of thifensulfuron hydrolase-1 (ALT-1) is shown as SEQ ID NO: 1 in the sequence listing; the ALT-1-01 nucleotide sequence (1197 nucleotides) encoding the corresponding ALT-1 amino acid sequence is shown as SEQ ID NO: 2 in the sequence listing; and the ALT-1-02 nucleotide sequence (1197 nucleotides) encoding the corresponding ALT-1 amino acid sequence is shown as SEQ ID NO: 3 in the sequence listing.

The amino acid sequence (369 amino acids) of thifensulfuron hydrolase-2 (ALT-2) is shown as SEQ ID NO: 4 in the sequence listing; the ALT-2-01 nucleotide sequence (1110 nucleotides) encoding the corresponding ALT-2 amino acid sequence is shown as SEQ ID NO: 5 in the sequence listing; and the ALT-2-02 nucleotide sequence (1110 nucleotides) encoding the corresponding ALT-2 amino acid sequence is shown as SEQ ID NO: 6 in the sequence listing.

The amino acid sequence (362 amino acids) of thifensulfuron hydrolase-3 (ALT-3) is shown as SEQ ID NO: 7 in the sequence listing; the ALT-3-01 nucleotide sequence (1089 nucleotides) encoding the corresponding ALT-3 amino acid sequence is shown as SEQ ID NO: 8 in the sequence listing; and the ALT-3-02 nucleotide sequence (1089 nucleotides) encoding the corresponding ALT-3 amino acid sequence is shown as SEQ ID NO: 9 in the sequence listing.

2. Acquisition of an EPSPS Gene Sequence

The amino acid sequence (455 amino acids) of a glyphosate-tolerant protein is shown as SEQ ID NO: 10 in the sequence listing; and the EPSPS nucleotide sequence (1368 nucleotides) encoding the amino acid sequence of the corresponding glyphosate-tolerant protein is shown as SEQ ID NO: 11 in the sequence listing.

3. Synthesis of the Above-Mentioned Nucleotide Sequences

The ALT-1-01 nucleotide sequence (shown as SEQ ID NO: 2 in the sequence listing), the ALT-1-02 nucleotide sequence (shown as SEQ ID NO: 3 in the sequence listing), the ALT-2-01 nucleotide sequence (shown as SEQ ID NO: 5 in the sequence listing), the ALT-2-02 nucleotide sequence (shown as SEQ ID NO: 6 in the sequence listing), the ALT-3-01 nucleotide sequence (shown as SEQ ID NO: 8 in the sequence listing), the ALT-3-02 nucleotide sequence (shown as SEQ ID NO: 9 in the sequence listing) and the EPSPS nucleotide sequence (shown as SEQ ID NO: 11 in the sequence listing) were synthesized by Nanjing Genscript Biotechnology Co., Ltd.; the synthetic ALT-1-01 nucleotide sequence (SEQ ID NO: 2) is further connected with a SpeI restriction site at the 5′ end, and the ALT-1-01 nucleotide sequence (SEQ ID NO: 2) is further connected with a KasI restriction site at the 3′ end; the synthetic ALT-1-02 nucleotide sequence (SEQ ID NO: 3) is further connected with a SpeI restriction site at the 5′ end, and the ALT-1-02 nucleotide sequence (SEQ ID NO: 3) is further connected with a KasI restriction site at the 3′ end; the synthetic ALT-2-01 nucleotide sequence (SEQ ID NO: 5) is further connected with a SpeI restriction site at the 5′ end, and the ALT-2-01 nucleotide sequence (SEQ ID NO: 5) is further connected with a KasI restriction site at the 3′ end; the synthetic ALT-2-02 nucleotide sequence (SEQ ID NO: 6) is further connected with a SpeI restriction site at the 5′ end, and the ALT-2-02 nucleotide sequence (SEQ ID NO: 6) is further connected with a KasI restriction site at the 3′ end; the synthetic ALT-3-01 nucleotide sequence (SEQ ID NO: 8) is further connected with a SpeI restriction site at the 5′ end, and the ALT-3-01 nucleotide sequence (SEQ ID NO: 8) is further connected with a KasI restriction site at the 3′ end; the synthetic ALT-3-02 nucleotide sequence (SEQ ID NO: 9) is further connected with a SpeI restriction site at the 5′ end, and the ALT-3-02 nucleotide sequence (SEQ ID NO: 9) is further connected with a KasI restriction site at the 3′ end; and the synthetic EPSPS nucleotide sequence (SEQ ID NO: 11) is further connected with a NcoI restriction site at the 5′ end, and the EPSPS nucleotide sequence (SEQ ID NO: 11) is further connected with a FspI restriction site at the 3′ end.

### Example 2. Construction of Arabidopsis thaliana Recombinant Expression Vectors

1. Construction of Arabidopsis thaliana and Soybean Recombinant Cloning Vectors Containing ALT Nucleotide Sequences

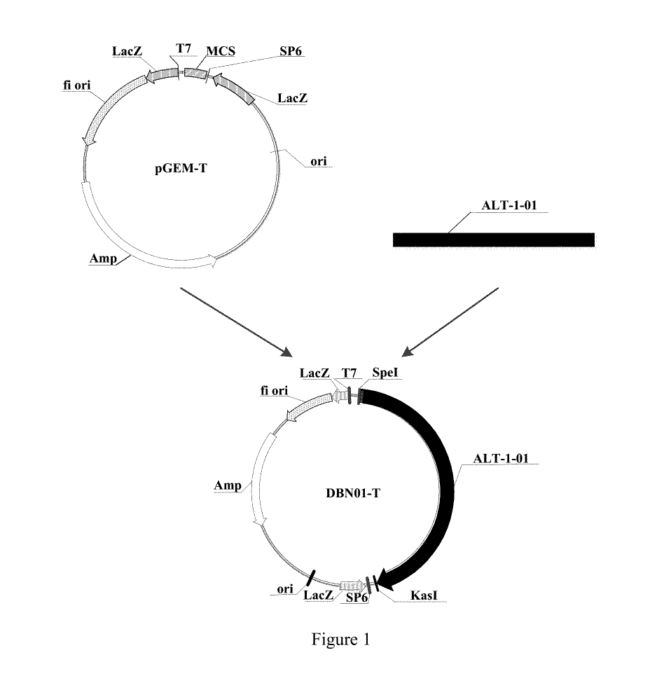

The synthetic ALT-1-01 nucleotide sequence was ligated into cloning vector pGEM-T (Promega, Madison, USA, CAT: A3600), and the operational procedure was carried out according to Promega's pGEM-T vector product instructions, obtaining a recombinant cloning vector DBN01-T, the construction process of which is as shown in FIG. 1 (wherein Amp means the ampicillin resistance gene; f1 means the origin of replication of phage f1; LacZ is LacZ initiation codon; SP6 is SP6 RNA polymerase promoter; T7 is T7 RNA polymerase promoter; ALT-1-01 is the ALT-1-01 nucleotide sequence (SEQ ID NO: 2); and MCS is a multiple cloning site).

Then, Escherichia coli T1 competent cells (Transgen, Beijing, China, CAT: CD501) were transformed with the recombinant cloning vector DBN01-T using the heat shock method with the following heat shock conditions: water bathing 50 μL Escherichia coli T1 competent cells and 10 μL plasmid DNA (recombinant cloning vector DBN01-T) at 42° C. for 30 seconds; shake culturing at 37° C. for 1 hour (using a shaker at a rotation speed of 100 rpm for shaking); and growing on an LB plate (10 g/L of tryptone, 5 g/L of yeast extract, 10 g/L of NaCl, and 15 g/L of agar, adjusting the pH to 7.5 with NaOH) of ampicillin (100 mg/L) having its surface coated with IPTG (isopropylthio-β-D-galactoside) and X-gal (5-bromo-4-chloro-3-indole-β-D-galactoside) overnight. White colonies were picked out and cultured in an LB liquid culture medium (10 g/L of tryptone, 5 g/L of yeast extract, 10 g/L of NaCl, and 100 mg/L of ampicillin, adjusting the pH to 7.5 with NaOH) at a temperature of 37° C. overnight. The plasmids in the cells were extracted through an alkaline method: centrifuging the bacteria solution at a rotation speed of 12000 rpm for 1 min, removing the supernatant, and suspending the precipitated thalli with 100 μL ice pre-cooled solution I (25 mM Tris-HCl, 10 mM EDTA (ethylenediaminetetraacetic acid), and 50 mM glucose, pH 8.0); adding 200 μL newly formulated solution II (0.2M NaOH, 1% SDS (sodium dodecyl sulfate)), inverting the tube 4 times, mixing and placing on ice for 3-5 min; adding 150 μL ice-cold solution III (3 M potassium acetate, 5 M acetic acid), mixing uniformly immediately and placing on ice for 5-10 min; centrifuging under the conditions of a temperature of 4° C. and a rotation speed of 12000 rpm for 5 min, adding 2 volumes of anhydrous ethanol to the supernatant and placing at room temperature for 5 min after mixing uniformly; centrifuging under the conditions of a temperature of 4° C. and a rotation speed of 12000 rpm for 5 min, discarding the supernatant, and air drying the precipitate after washing with ethanol with a concentration of 70% (V/V); adding 30 μL TE (10 mM Tris-HCl, and 1 mM EDTA, pH 8.0) containing RNase (20 μg/mL) to dissolve the precipitate; water bathing at a temperature of 37° C. for 30 min to digest RNA; and storing at a temperature of −20° C. for use.

After identifying the extracted plasmid by SpeI and KasI digestion, positive clones were verified by sequencing. The results showed that the inserted ALT-1-01 nucleotide sequence in the recombinant cloning vector DBN01-T was the nucleotide sequence shown as SEQ ID NO: 2 in the sequence listing, that is, the ALT-1-01 nucleotide sequence was inserted correctly.

According to the above-mentioned method for constructing the recombinant cloning vector DBN01-T, the synthetic ALT-2-01 nucleotide sequence was ligated into a cloning vector pGEM-T, obtaining a recombinant cloning vector DBN02-T, wherein ALT-2-01 is the ALT-2-01 nucleotide sequence (SEQ ID NO: 5). Enzyme digestion and sequencing verified that the ALT-2-01 nucleotide sequence was correctly inserted into the recombinant cloning vector DBN02-T.

According to the above-mentioned method for constructing the recombinant cloning vector DBN01-T, the synthetic ALT-3-01 nucleotide sequence was ligated into a cloning vector pGEM-T, obtaining a recombinant cloning vector DBN03-T, wherein ALT-3-01 is the ALT-3-01 nucleotide sequence (SEQ ID NO: 8). Enzyme digestion and sequencing verified that the ALT-3-01 nucleotide sequence was correctly inserted into the recombinant cloning vector DBN03-T.

At the same time, according to the above-mentioned method for constructing the recombinant cloning vector DBN01-T, the synthetic EPSPS nucleotide sequence was ligated into a cloning vector pGEM-T, obtaining a recombinant cloning vector DBN04-T, wherein EPSPS is the EPSPS nucleotide sequence (SEQ ID NO: 11). Enzyme digestion and sequencing verified that the EPSPS nucleotide sequence was correctly inserted into the recombinant cloning vector DBN04-T.

2. Construction of Arabidopsis thaliana Recombinant Expression Vectors Containing ALT Nucleotide Sequences

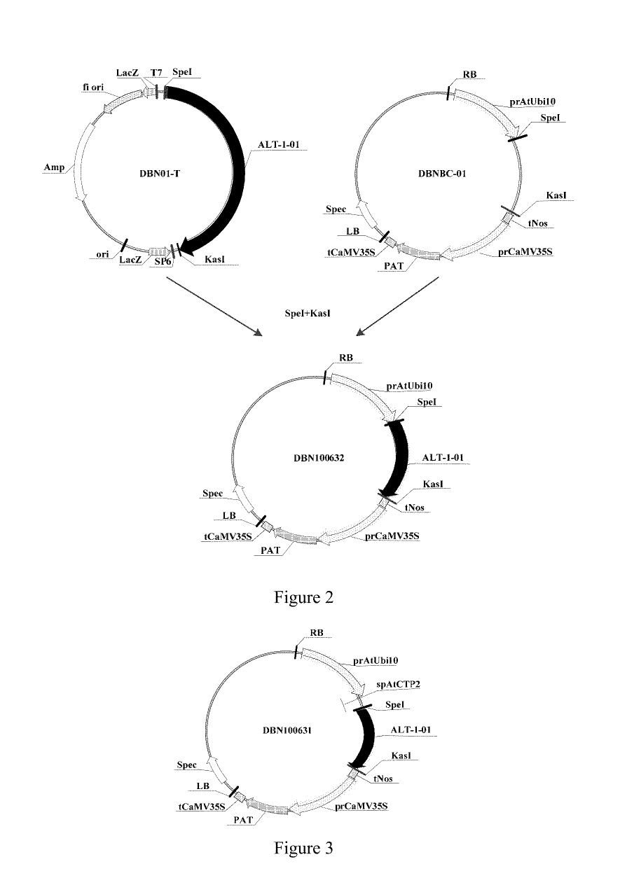

The recombinant cloning vector DBN01-T and an expression vector DBNBC-01 (vector backbone: pCAMBIA2301 (which can be provided by the CAMBIA institution)) were digested with restriction enzymes SpeI and KasI, respectively; the excised ALT-1-01 nucleotide sequence fragment was inserted between the SpeI and KasI sites in the expression vector DBNBC-01; and it is well known to a person skilled in the art to construct a vector using conventional enzyme digestion methods, a recombinant expression vector DBN100632 was constructed (located in the cytoplasm), and the construction process of which was shown as FIG. 2 (Spec: the spectinomycin gene; RB: the right boundary; prAtUbi10: the Arabidopsis thaliana Ubiquitin 10 gene promoter (SEQ ID NO: 12); ALT-1-01: the ALT-1-01 nucleotide sequence (SEQ ID NO: 2); tNos: the terminator of nopaline synthase gene (SEQ ID NO:13); prCaMV35S: the cauliflower mosaic virus 35S promoter (SEQ ID NO: 14); PAT: the glufosinate acetyltransferase gene (SEQ ID NO: 15); tCaMV35S: the cauliflower mosaic virus 35S terminator (SEQ ID NO: 16); LB: the left boundary).

Escherichia coli T1 competent cells were transformed with the recombinant expression vector DBN100632 by a heat shock method with the following heat shock conditions: water bathing 50 μL Escherichia coli T1 competent cells and 10 μL plasmid DNA (recombinant expression vector DBN100632) at 42° C. for 30 seconds; shake culturing at 37° C. for 1 hour (using a shaker at a rotation speed of 100 rpm for shaking); then culturing under the condition of a temperature of 37° C. on an LB solid plate containing 50 mg/L of spectinomycin (10 g/L of tryptone, 5 g/L of yeast extract, 10 g/L of NaCl, and 15 g/L of agar, adjusted to a pH of 7.5 with NaOH) for 12 hours, picking white colonies, and culturing under the condition of a temperature of 37° C. overnight in an LB liquid culture medium (10 g/L of tryptone, 5 g/L of yeast extract, 10 g/L of NaCl, and 50 mg/L of spectinomycin, adjusted to a pH of 7.5 with NaOH). The plasmids in the cells were extracted through an alkaline method. The extracted plasmid was identified after digesting with restriction enzymes SpeI and KasI, and positive clones were identified by sequencing. The results showed that the nucleotide sequence between the SpeI and KasI sites in the recombinant expression vector DBN100632 was the nucleotide sequence shown as SEQ ID NO: 2 in the sequence listing, i.e., the ALT-1-01 nucleotide sequence.

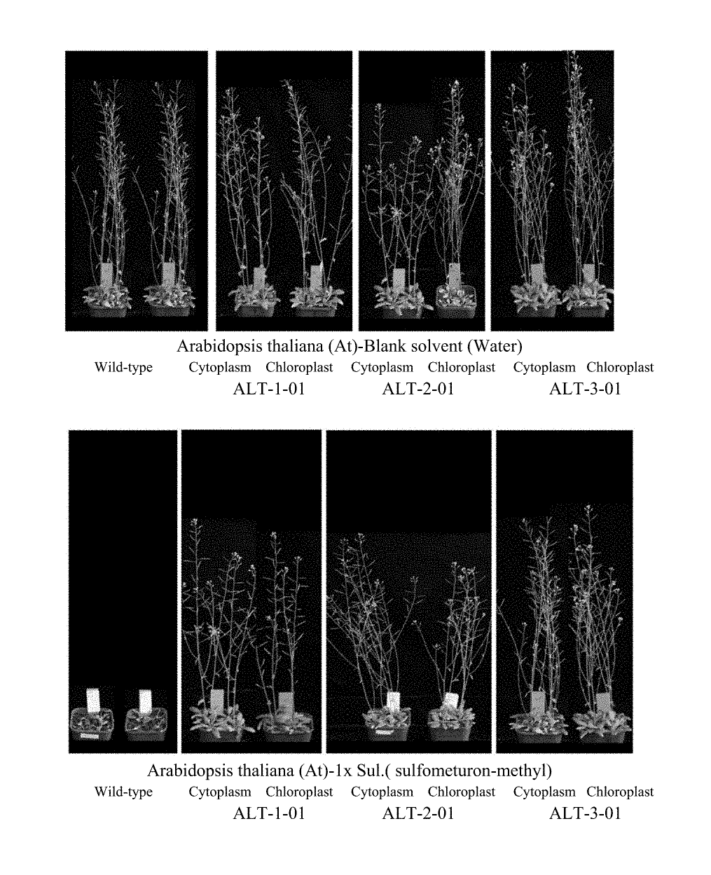

According to the above-mentioned method for constructing the recombinant expression vector DBN100632, a recombinant expression vector DBN100631 (located in the chloroplast) containing the ALT-1-01 nucleotide sequence was constructed, the vector structure of which was shown as FIG. 3 (vector backbone: pCAMBIA2301 (which can be provided by the CAMBIA institution); Spec: the spectinomycin gene; RB: the right boundary; prAtUbi10: the Arabidopsis thaliana Ubiquitin 10 gene promoter (SEQ ID NO: 12); spAtCTP2: the Arabidopsis thaliana chloroplast transit peptide (SEQ ID NO: 17); ALT-1-01: the ALT-1-01 nucleotide sequence (SEQ ID NO: 2); tNos: the terminator of nopaline synthase gene (SEQ ID NO:13); prCaMV35S: the cauliflower mosaic virus 35S promoter (SEQ ID NO: 14); PAT: the glufosinate acetyltransferase gene (SEQ ID NO: 15); tCaMV35S: the cauliflower mosaic virus 35S terminator (SEQ ID NO: 16); LB: the left boundary). Positive clones were verified by sequencing. The results showed that the inserted ALT-1-01 nucleotide sequence in the recombinant expression vector DBN100631 was the nucleotide sequence shown as SEQ ID NO: 2 in the sequence listing, that is, the ALT-1-01 nucleotide sequence was inserted correctly.

According to the above-mentioned method for constructing the recombinant expression vector DBN100632, the ALT-2-01 nucleotide sequence excised by SpeI and KasI digested recombinant cloning vector DBN02-T was inserted into the expression vector DBNBC-01, obtaining a recombinant expression vector DBN100634. Enzyme digestion and sequencing verified that the nucleotide sequence in the recombinant expression vector DBN100634 contained the nucleotide sequence shown as SEQ ID NO: 5 in the sequence listing, that is, the ALT-2-01 nucleotide sequence was inserted correctly.

According to the above-mentioned method for constructing the recombinant expression vector DBN100631, the ALT-2-01 nucleotide sequence excised by SpeI and KasI digested recombinant cloning vector DBN02-T was inserted into the expression vector DBNBC-01, obtaining a recombinant expression vector DBN100633 (containing spAtCTP2, located in the chloroplast). Enzyme digestion and sequencing verified that the nucleotide sequence in the recombinant expression vector DBN100633 contained the nucleotide sequence shown as SEQ ID NO: 5 in the sequence listing, that is, the ALT-2-01 nucleotide sequence was inserted correctly.

According to the above-mentioned method for constructing the recombinant expression vector DBN100632, the ALT-3-01 nucleotide sequence excised by SpeI and KasI digested recombinant cloning vector DBN03-T was inserted into the expression vector DBNBC-01, obtaining a recombinant expression vector DBN100636. Enzyme digestion and sequencing verified that the nucleotide sequence in the recombinant expression vector DBN100636 contained the nucleotide sequence shown as SEQ ID NO: 8 in the sequence listing, that is, the ALT-3-01 nucleotide sequence was inserted correctly.

According to the above-mentioned method for constructing the recombinant expression vector DBN100631, the ALT-3-01 nucleotide sequence excised by SpeI and KasI digested recombinant cloning vector DBN03-T was inserted into the expression vector DBNBC-01, obtaining a recombinant expression vector DBN100635 (containing spAtCTP2, located in the chloroplast). Enzyme digestion and sequencing verified that the nucleotide sequence in the recombinant expression vector DBN100635 contained the nucleotide sequence shown as SEQ ID NO: 8 in the sequence listing, that is, the ALT-3-01 nucleotide sequence was inserted correctly.

### Example 3. Acquisition of Arabidopsis thaliana Plants Having an ALT Nucleotide Sequence Introduced

1. Transformation of Agrobacterium with the Recombinant Expression Vectors

The Agrobacterium GV3101 was transformed with the recombinant expression vectors DBN100632, DBN100631, DBN100634, DBN100633, DBN100636 and DBN100635 which had been correctly constructed using the liquid nitrogen method with the following transformation conditions: placing 100 μL of Agrobacterium GV3101, and 3 μL of plasmid DNA (recombinant expression vector) in liquid nitrogen for 10 minutes, warm water bathing at 37° C. for 10 minutes; inoculating the transformed Agrobacterium GV3101 into an LB tube, culturing under the conditions of a temperature of 28° C. and a rotation speed of 200 rpm for 2 hours, spreading on an LB plate containing 50 mg/L of rifampicin and 50 mg/L of spectinomycin until positive single clones were grown, picking out single clones for culturing and extracting the plasmids thereof, and performing enzyme digestion verification using restriction enzymes. The results showed that the structures of the recombinant expression vectors DBN100632, DBN100631, DBN100634, DBN100633, DBN100636 and DBN100635 were completely correct.

2. Acquisition of Transgenic Arabidopsis thaliana Plants

Seeds of wild-type Arabidopsis thaliana were suspended in a 0.1% (w/v) agarose solution. The suspended seeds were stored at 4° C. for 2 days to complete the need for dormancy, in order to ensure synchronous seed germination. Vermiculite was mixed with horse manure soil, the mixture was sub-irrigated with water to wet, and the soil mixture was allowed to drain the water away for 24 hours. The pretreated seeds were sowed in the soil mixture and covered with a moisturizing cover for 7 days. The seeds were germinated and the plants were cultivated in a greenhouse under long day conditions (16 hours of light/8 hours of dark) of a constant temperature (22° C.) and a constant humidity (40-50%) with a light intensity of 120-150 μmol/(m²·sec). The plants were initially irrigated with the Hoagland's nutrient solution, followed by deionized water, keeping the soil moist but not wet through.

Arabidopsis thaliana was transformed using the flower soaking method. One or more 15-30 mL of precultures of YEP culture solution (containing spectinomycin (50 mg/L) and rifampicin (10 mg/L)) were inoculated with the selected Agrobacterium colonies. The cultures were incubated at 28° C. and 220 rpm with shaking at a constant speed overnight. Each preculture was used to inoculate two 500 mL of cultures of YEP culture solution (containing spectinomycin (50 mg/L) and rifampicin (10 mg/L)), and the cultures were incubated at 28° C. with continuous shaking overnight. Cells were precipitated by centrifuging at about 8700×g at room temperature for 10 minutes, and the resulting supernatant was discarded. The cell precipitate was gently re-suspended in 500 mL osmotic medium which contained ½×MS salt/B5 vitamin, 10% (w/v) sucrose, 0.044 μM benzylaminopurine (10 μL/L (1 mg/mL, a stock solution in DMSO)) and 300 μL/L of Silvet L-77. About 1-month-old plants were soaked in a culture medium for 15 seconds to ensure immersion of the latest inflorescence. Then, the plants were reclined laterally and covered (transparently or opaquely) for 24 hours, then washed with water, and placed vertically. The plants were cultivated with a photoperiod of 16 hours of light/8 hours of dark at 22° C. Seeds were harvested after soaking for about 4 weeks.

The newly harvested (ALT nucleotide sequence) T₁ seeds were dried at room temperature for 7 days. The seeds were sowed in 26.5×51 cm germination disks, and 200 mg T₁ seeds (about 10000 seeds) were accepted per disk, wherein the seeds had been previously suspended in 40 mL of 0.1% (w/v) agarose solution and stored at 4° C. for 2 days to complete the need for dormancy, in order to ensure synchronous seed germination.

Vermiculite was mixed with horse manure soil, the mixture was sub-irrigated with water to wet, and water was drained through gravity. The pretreated seeds (each 40 mL) were sowed evenly in the soil mixture using a pipette, and covered with a moisturizing cover for 4-5 days. The cover was removed 1 day before performing initial transformant selection by spraying glufosinate (used to select the co-transformed PAT gene) post emergence.

The T₁ plants were sprayed with a 0.2% solution of a Liberty herbicide (200 g ai/L of glufosinate) reusing a DeVilbiss compressed air nozzle at a spray volume of 10 mL/disc (703 L/ha) 7 days after planting (DAP) and 11 DAP (the cotyledon stage and 2-4 leaf stage, respectively), to provide an effective amount of glufosinate of 280 g ai/ha per application. Surviving plants (actively growing plants) were identified 4-7 days after the final spraying, and transplanted to 7 cm×7 cm square pots prepared with horse manure soil and vermiculite (3-5 plants/disc), respectively. The transplanted plants were covered with a moisturizing cover for 3-4 days, and placed in a 22° C. culture chamber or directly transferred into a greenhouse as previously. Then, the cover was removed, and at least 1 day before testing the ability of the ALT gene to provide Sulfometuron-methyl herbicide resistance, the plants were planted into a greenhouse (22±5° C., 50±30% RH, 14 hours of light: 10 hours of dark, a minimum of 500 μE/m²s¹ natural+supplemental light).

### Example 4. Detection of Herbicide Tolerance Effects of the Transgenic Arabidopsis thaliana Plants

T₁ transformants were initially selected from the background of untransformed seeds using a glufosinate selection scheme. About 40000 T₁ seeds were screened, and 380 T₁ positive transformants (PAT gene) were identified with a transformation efficiency of about 0.95%. The plants that were transformed with the recombinant expression vector DBN100632 were Arabidopsis thaliana plants having an ALT-1-01 nucleotide sequence located in the cytoplasm introduced (At cytoplasmic ALT-1-01), and the plants that were transformed with the recombinant expression vector DBN100631 were Arabidopsis thaliana plants having an ALT-1-01 nucleotide sequence located in the chloroplast introduced (At chloroplastic ALT-1-01); the plants that were transformed with the recombinant expression vector DBN100634 were Arabidopsis thaliana plants having an ALT-2-01 nucleotide sequence located in the cytoplasm introduced (At cytoplasmic ALT-2-01), and the plants that were transformed with the recombinant expression vector DBN100633 were Arabidopsis thaliana plants having an ALT-2-01 nucleotide sequence located in the chloroplast introduced (At chloroplastic ALT-2-01); and the plants that were transformed with the recombinant expression vector DBN100636 were Arabidopsis thaliana plants having an ALT-3-01 nucleotide sequence located in the cytoplasm introduced (At cytoplasmic ALT-3-01), and the plants that were transformed with the recombinant expression vector DBN100635 were Arabidopsis thaliana plants having an ALT-3-01 nucleotide sequence located in the chloroplast introduced (At chloroplastic ALT-3-01). The herbicide tolerance effects of At cytoplasmic ALT-1-01 T₁ plants, At chloroplastic ALT-1-01 T₁ plants, At cytoplasmic ALT-2-01 T₁ plants, At chloroplastic ALT-2-01 T₁ plants, At cytoplasmic ALT-3-01 T₁ plants, At chloroplastic ALT-3-01 T₁ plants and wild-type Arabidopsis thaliana plants on Sulfometuron-methyl were detected (14 days after sowing), respectively.

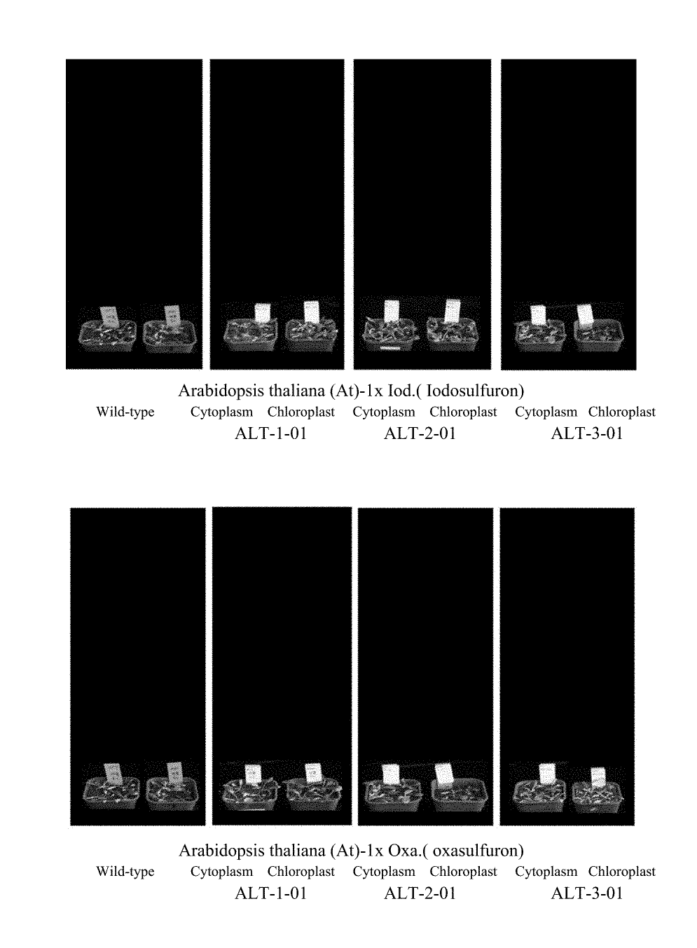

At cytoplasmic ALT-1-01 T₁ plants, At chloroplastic ALT-1-01 T₁ plants, At cytoplasmic ALT-2-01 T₁ plants, At chloroplastic ALT-2-01 T₁ plants, At cytoplasmic ALT-3-01 T₁ plants, At chloroplastic ALT-3-01 T₁ plants and wild-type Arabidopsis thaliana plants were sprayed with Sulfometuron-methyl (30 g ai/ha, 1-fold field concentration) and a blank solvent (water), respectively. Plants were counted for the resistance situations 14 days after spraying: those having a consistent growth status with the blank solvent (water) group after 14 days were classified as highly resistant plants, those having a bolting height less than ½ of that of the blank solvent (water) group after 14 days were classified as moderately resistant plants, those still not capable of bolting after 14 days were classified as poorly resistant plants, and those dead after 14 days were classified as non-resistant plants. Since each Arabidopsis thaliana T₁ plant was an independent transformation event, a significant difference in individual T1 responses could be expected at a given dose. The results are as shown in Table 1 and FIG. 4.

TABLE 1
Experimental results of the tolerance of transgenic Arabidopsis
thaliana T₁ plants to a Sulfometuron-methyl herbicide

| | Arabidopsis | | Moder- | | | |
|---|---|---|---|---|---|---|
| | thaliana | Highly | ately | Poorly | Non- | |
| Treatment | genotypes | resistant | resistant | resistant | resistant | Total |
| Blank | At | 30 | 0 | 0 | 0 | 30 |
| solvent | cytoplasmic | | | | | |
| (water) | ALT-1-01 | | | | | |
| | At | 28 | 0 | 0 | 0 | 28 |
| | chloroplastic | | | | | |
| | ALT-1-01 | | | | | |
| | At | 31 | 0 | 0 | 0 | 31 |
| | cytoplasmic | | | | | |
| | ALT-2-01 | | | | | |
| | At | 25 | 0 | 0 | 0 | 25 |
| | chloroplastic | | | | | |
| | ALT-2-01 | | | | | |
| | At | 27 | 0 | 0 | 0 | 27 |
| | cytoplasmic | | | | | |
| | ALT-3-01 | | | | | |
| | At | 27 | 0 | 0 | 0 | 27 |
| | chloroplastic | | | | | |
| | ALT-3-01 | | | | | |
| | wild-type | 30 | 0 | 0 | 0 | 30 |
| 30 g ai/ha | At | 23 | 2 | 1 | 1 | 27 |
| Sulfo- | cytoplasmic | | | | | |
| meturon- | ALT-1-01 | | | | | |
| methyl | At | 27 | 0 | 0 | 1 | 28 |
| (1x Tri.) | chloroplastic | | | | | |
| | ALT-1-01 | | | | | |
| | At | 24 | 0 | 2 | 3 | 29 |
| | cytoplasmic | | | | | |
| | ALT-2-01 | | | | | |
| | At | 29 | 0 | 1 | 0 | 30 |
| | chloroplastic | | | | | |
| | ALT-2-01 | | | | | |
| | At | 22 | 1 | 1 | 3 | 27 |
| | cytoplasmic | | | | | |
| | ALT-3-01 | | | | | |
| | At | 27 | 0 | 0 | 1 | 28 |
| | chloroplastic | | | | | |
| | ALT-3-01 | | | | | |
| | wild-type | 0 | 0 | 0 | 32 | 32 |

For Arabidopsis thaliana, 30 g ai/ha Sulfometuron-methyl herbicide is an effective dose distinguishing sensitive plants from plants having an average level of resistance. The results of Table 1 and FIG. 4 show that: the thifensulfuron hydrolase (ALT-1, ALT-2 and ALT-3) conferred Sulfometuron-methyl herbicide tolerance to individual Arabidopsis thaliana plants (the reason why individual plants were not tolerant was that the insertion site in the T₁ plants was random, the expression levels of the tolerance gene were different, showing a difference in tolerance level); compared to At cytoplasmic ALT-1-01 T₁ plants, At cytoplasmic ALT-2-01 T₁ plants and At cytoplasmic ALT-3-01 T₁ plants, At chloroplastic ALT-1-01 T₁ plants, At chloroplastic ALT-2-01 T₁ plants and At chloroplastic ALT-3-01 T₁ plants were able to produce a higher Sulfometuron-methyl herbicide tolerance, suggesting that the thifensulfuron hydrolase (ALT-1, ALT-2 and ALT-3) gene may enhance the tolerance of Arabidopsis thaliana plants to the Sulfometuron-methyl herbicide when located in the chloroplast for expression; while none of the wild-type Arabidopsis thaliana plants was tolerant to the Sulfometuron-methyl herbicide.

### Example 5. Having an Unexpected Technical Effect on Different Sulfonylurea Herbicides

The thifensulfuron hydrolase, which can also be known as sulfonylurea herbicide de-esterase, degrades ester bond-containing sulfonylurea herbicides (e.g., thifensulfuron, etc.) into herbicidally inactive mother acids by hydrolyzing the ester bond, and therefore it cannot degrade ester bond-free sulfonylurea herbicides (e.g., nicosulfuron, chlorsulfuron, etc.). In the prior art, there are many sulfonylurea herbicides containing ester bonds and having similar structures, such as Sulfometuron-methyl, iodosulfuron-methyl, oxasulfuron, mesosulfuron (mesosulfuron-methyl), pyrazosulfuron-ethyl, sulfometuron-methyl, and halosulfuron-methyl.

At cytoplasmic ALT-1-01 T₁ plants, At chloroplastic ALT-1-01 T₁ plants, At cytoplasmic ALT-2-01 T₁ plants, At chloroplastic ALT-2-01 T₁ plants, At cytoplasmic ALT-3-01 T₁ plants, At chloroplastic ALT-3-01 T₁ plants and wild-type Arabidopsis thaliana plants in Example 4 were sprayed with iodosulfuron-methyl (10 g ai/ha, 1-fold field concentration), mesosulfuron (14 g ai/ha, 1-fold field concentration) and oxasulfuron (60 g ai/ha, 1-fold field concentration), respectively, in addition to Sulfometuron-methyl (30 g ai/ha, 1-fold field concentration) and the blank solvent (water). Plants were counted for the resistance situation 14 days after spraying: those having a consistent growth status with the blank solvent (water) group after 14 days were classified as highly resistant plants, those having a bolting height less than ½ of that of the blank solvent (water) group after 14 days were classified as moderately resistant plants, those still not capable of bolting after 14 days were classified as poorly resistant plants, and those dead after 14 days were classified as non-resistant plants. Since each Arabidopsis thaliana T₁ plant was an independent transformation event, a significant difference in individual T₁ responses could be expected at a given dose. The results are as shown in Table 2 and FIG. 4.

TABLE 2
Experimental results of the tolerance of transgenic Arabidopsis
thaliana T₁ plants to sulfonylurea herbicides

| | Arabidopsis | | Moder- | | | |
|---|---|---|---|---|---|---|
| | thaliana | Highly | ately | Poorly | Non- | |
| Treatment | genotypes | resistant | resistant | resistant | resistant | Total |
| Blank | At cytoplasmic | 30 | 0 | 0 | 0 | 30 |
| solvent | ALT-1-01 | | | | | |
| (water) | At | 28 | 0 | 0 | 0 | 28 |
| | chloroplastic | | | | | |
| | ALT-1-01 | | | | | |
| | At cytoplasmic | 31 | 0 | 0 | 0 | 31 |
| | ALT-2-01 | | | | | |
| | At | 25 | 0 | 0 | 0 | 25 |
| | chloroplastic | | | | | |
| | ALT-2-01 | | | | | |
| | At cytoplasmic | 27 | 0 | 0 | 0 | 27 |
| | ALT-3-01 | | | | | |
| | At | 27 | 0 | 0 | 0 | 27 |
| | chloroplastic | | | | | |
| | ALT-3-01 | | | | | |
| | wild-type | 30 | 0 | 0 | 0 | 30 |
| 10 g ai/ha | At cytoplasmic | 0 | 0 | 0 | 29 | 29 |
| iodo- | ALT-1-01 | | | | | |
| sulfuron- | At | 0 | 0 | 0 | 30 | 30 |
| methyl | chloroplastic | | | | | |
| (1x Iod.) | ALT-1-01 | | | | | |
| | At cytoplasmic | 0 | 0 | 0 | 30 | 30 |
| | ALT-2-01 | | | | | |
| | At | 0 | 0 | 0 | 31 | 31 |
| | chloroplastic | | | | | |
| | ALT-2-01 | | | | | |
| | At cytoplasmic | 0 | 0 | 0 | 30 | 30 |
| | ALT-3-01 | | | | | |
| | At | 0 | 0 | 0 | 32 | 32 |
| | chloroplastic | | | | | |
| | ALT-3-01 | | | | | |
| | wild-type | 0 | 0 | 0 | 29 | 29 |
| 14 g ai/ha | At cytoplasmic | 0 | 0 | 0 | 29 | 29 |
| meso- | ALT-1-01 | | | | | |
| sulfuron | At | 0 | 0 | 0 | 32 | 32 |
| (1x Mes.) | chloroplastic | | | | | |
| | ALT-1-01 | | | | | |
| | At cytoplasmic | 0 | 0 | 0 | 32 | 32 |
| | ALT-2-01 | | | | | |
| | At | 0 | 0 | 0 | 30 | 30 |
| | chloroplastic | | | | | |
| | ALT-2-01 | | | | | |
| | At cytoplasmic | 0 | 0 | 0 | 30 | 30 |
| | ALT-3-01 | | | | | |
| | At | 0 | 0 | 0 | 32 | 32 |
| | chloroplastic | | | | | |
| | ALT-3-01 | | | | | |
| | wild-type | 0 | 0 | 0 | 28 | 28 |
| 60 g ai/ha | At cytoplasmic | 0 | 0 | 0 | 28 | 28 |
| oxa- | ALT-1-01 | | | | | |
| sulfuron | At | 0 | 0 | 0 | 30 | 30 |
| (1x Oxa.) | chloroplastic | | | | | |
| | ALT-1-01 | | | | | |
| | At cytoplasmic | 0 | 0 | 0 | 30 | 30 |
| | ALT-2-01 | | | | | |
| | At | 0 | 0 | 0 | 30 | 30 |
| | chloroplastic | | | | | |
| | ALT-2-01 | | | | | |
| | At cytoplasmic | 0 | 0 | 0 | 30 | 30 |
| | ALT-3-01 | | | | | |
| | At | 0 | 0 | 0 | 31 | 31 |
| | chloroplastic | | | | | |
| | ALT-3-01 | | | | | |
| | wild-type | 0 | 0 | 0 | 28 | 28 |

The responses of inputting the thifensulfuron hydrolase activity to Arabidopsis thaliana T₁ plants by ALT-1, ALT-2 and ALT-3 were compared in Table 2. The thifensulfuron hydrolase activity was conferred to all the transformed Arabidopsis thaliana T₁ plants; however, in the given treatments (iodosulfuron-methyl, mesosulfuron and oxasulfuron), all the transformed Arabidopsis thaliana T₁ plants did not exhibit the ability to degrade the above-mentioned sulfonylurea herbicides, and there was no difference between all the transformed Arabidopsis thaliana T₁ plants (ALT-1, ALT-2 and ALT-3) and the wild-type Arabidopsis thaliana plants in the degree of damage.

Table 2 fully illustrated that the results of Table 1 were unexpected. Although Sulfometuron-methyl as well as thifensulfuron, iodosulfuron-methyl, mesosulfuron and oxasulfuron are all sulfonylurea herbicides containing ester bonds and having similar chemical structures, the given treatments were also comparable (1-fold field concentration) and at the same time, the thifensulfuron hydrolase (ALT-1, ALT-2 and ALT-3) had been input and expressed at an expected level in the plant individuals, plants expressing the thifensulfuron hydrolase neither had the ability to degrade iodosulfuron-methyl, mesosulfuron and oxasulfuron, nor could protect themselves from damage from the above-mentioned sulfonylurea herbicides, and showed no difference from the wild-type plants in performance, wherein these data are sufficient to confirm that the Sulfometuron-methyl herbicide tolerance conferred by the thifensulfuron hydrolase (ALT-1, ALT-2 and ALT-3) on the plants was difficult to predict.

### Example 6. Construction of Soybean Recombinant Expression Vectors and Transformation of Agrobacterium with the Recombinant Expression Vectors

1. Construction of Soybean Recombinant Expression Vectors Containing ALT Nucleotide Sequences

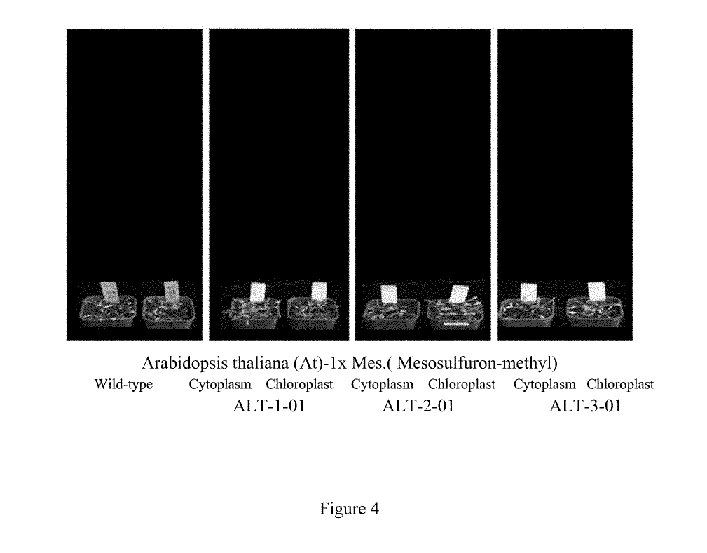

The recombinant cloning vectors DBN01-T and DBN04-T as well as an expression vector DBNBC-02 (vector backbone: pCAMBIA2301 (which can be provided by the CAMBIA institution)) were digested with restriction enzymes SpeI and KasI as well as NcoI and FspI, respectively; the excised ALT-1-01 nucleotide sequence and EPSPS nucleotide sequence fragments were inserted between the SpeI and KasI as well as NcoI and FspI sites in the expression vector DBNBC-02, respectively; and it is well known to a person skilled in the art to construct a vector using conventional enzyme digestion methods, a recombinant expression vector DBN100828 was constructed (located in the cytoplasm), the construction process of which is as shown in FIG. 5 (Spec: the spectinomycin gene; RB: the right boundary; prAtUbi10: the Arabidopsis thaliana Ubiquitin 10 gene promoter (SEQ ID NO: 12); ALT-1-01: the ALT-1-01 nucleotide sequence (SEQ ID NO: 2); tNos: the terminator of nopaline synthase gene (SEQ ID NO:13); prBrCBP: the rape eukaryotic elongation factor gene 1α (Tsf1) promoter (SEQ ID NO: 18); spAtCTP2: the Arabidopsis thaliana chloroplast transit peptide (SEQ ID NO: 17); EPSPS: the 5-enolpyruvylshikimate-3-phosphate synthase gene (SEQ ID NO: 11); tPsE9: the pea RbcS gene terminator (SEQ ID NO: 19); LB: the left boundary).

According to the method in point 2 of Example 2, Escherichia coli T1 competent cells were transformed with the recombinant expression vector DBN100828 using the heat shock method, and the plasmids in the cells were extracted through the alkaline method. The extracted plasmid was identified after digesting with restriction enzymes SpeI and KasI, and positive clones were identified by sequencing. The results showed that the nucleotide sequence between the SpeI and KasI sites in the recombinant expression vector DBN100828 was the nucleotide sequence shown as SEQ ID NO: 2 in the sequence listing, i.e., the ALT-1-01 nucleotide sequence.

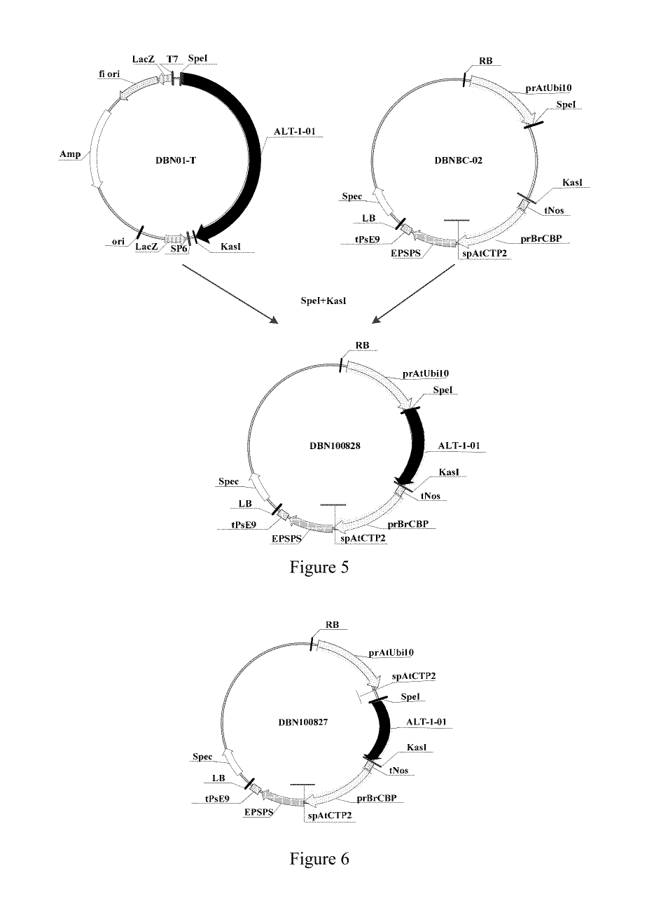

According to the above-mentioned method for constructing the recombinant expression vector DBN100828, a recombinant expression vector DBN100827 (located in the chloroplast) containing the ALT-1-01 nucleotide sequence was constructed, the vector structure of which was shown as FIG. 6 (vector backbone: pCAMBIA2301 (which can be provided by the CAMBIA institution); Spec: the spectinomycin gene; RB: the right boundary; prAtUbi10: the Arabidopsis thaliana Ubiquitin 10 gene promoter (SEQ ID NO: 12); spAtCTP2: the Arabidopsis thaliana chloroplast transit peptide (SEQ ID NO: 17); ALT-1-01: the ALT-1-01 nucleotide sequence (SEQ ID NO: 2); tNos: the terminator of nopaline synthase gene (SEQ ID NO:13); prBrCBP: the rape eukaryotic elongation factor gene 1α (Tsf1) promoter (SEQ ID NO: 18); spAtCTP2: the Arabidopsis thaliana chloroplast transit peptide (SEQ ID NO: 17); EPSPS: the 5-enolpyruvylshikimate-3-phosphate synthase gene (SEQ ID NO: 11); tPsE9: the pea RbcS gene terminator (SEQ ID NO: 19); LB: the left boundary). Positive clones were verified by sequencing. The results showed that the inserted ALT-1-01 nucleotide sequence in the recombinant expression vector DBN100827 was the nucleotide sequence shown as SEQ ID NO: 2 in the sequence listing, that is, the ALT-1-01 nucleotide sequence was inserted correctly.

According to the above-mentioned method for constructing the recombinant expression vector DBN100828, the ALT-2-01 nucleotide sequence and the EPSPS nucleotide sequence excised by SpeI and KasI as well as NcoI and FspI from digested recombinant cloning vectors DBN02-T and DBN04-T were inserted into the expression vector DBNBC-02, obtaining a recombinant expression vector DBN100826. Enzyme digestion and sequencing verified that the nucleotide sequences in the recombinant expression vector DBN100826 contained the nucleotide sequences shown as SEQ ID NO: 5 and SEQ ID NO: 11 in the sequence listing, that is, the ALT-2-01 nucleotide sequence and the EPSPS nucleotide sequence were inserted correctly.

According to the above-mentioned method for constructing the recombinant expression vector DBN100827, the ALT-2-01 nucleotide sequence and the EPSPS nucleotide sequence excised by SpeI and KasI as well as NcoI and FspI from digested recombinant cloning vectors DBN02-T and DBN04-T were inserted into the expression vector DBNBC-02, obtaining a recombinant expression vector DBN100825 (containing spAtCTP2, located in the chloroplast). Enzyme digestion and sequencing verified that the nucleotide sequences in the recombinant expression vector DBN100825 contained the nucleotide sequences shown as SEQ ID NO: 5 and SEQ ID NO: 11 in the sequence listing, that is, the ALT-2-01 nucleotide sequence and the EPSPS nucleotide sequence were inserted correctly.

According to the above-mentioned method for constructing the recombinant expression vector DBN100828, the ALT-3-01 nucleotide sequence and the EPSPS nucleotide sequence excised by SpeI and KasI as well as NcoI and FspI from digested recombinant cloning vectors DBN03-T and DBN04-T were inserted into the expression vector DBNBC-02, obtaining a recombinant expression vector DBN100824. Enzyme digestion and sequencing verified that the nucleotide sequences in the recombinant expression vector DBN100824 contained the nucleotide sequences shown as SEQ ID NO: 8 and SEQ ID NO: 11 in the sequence listing, that is, the ALT-3-01 nucleotide sequence and the EPSPS nucleotide sequence were inserted correctly.

According to the above-mentioned method for constructing the recombinant expression vector DBN100827, the ALT-3-01 nucleotide sequence and the EPSPS nucleotide sequence excised by SpeI and KasI as well as NcoI and FspI from digested recombinant cloning vectors DBN03-T and DBN04-T were inserted into the expression vector DBNBC-02, obtaining a recombinant expression vector DBN100823 (containing spAtCTP2, located in the chloroplast). Enzyme digestion and sequencing verified that the nucleotide sequences in the recombinant expression vector DBN100823 contained the nucleotide sequences shown as SEQ ID NO: 8 and SEQ ID NO: 11 in the sequence listing, that is, the ALT-3-01 nucleotide sequence and the EPSPS nucleotide sequence were inserted correctly.

2. Transformation of Agrobacterium with the recombinant expression vectors Agrobacterium LBA4404 (Invitrogen, Chicago, USA, CAT: 18313-015) was transformed with the recombinant expression vectors DBN100828, DBN100827, DBN100826, DBN100825, DBN100824 and DBN100823 which had been correctly constructed using a liquid nitrogen method, with the following transformation conditions: placing 100 μL of Agrobacterium LBA4404, and 3 μL of plasmid DNA (recombinant expression vector) in liquid nitrogen for 10 minutes, warm water bathing at 37° C. for 10 minutes; inoculating the transformed Agrobacterium LBA4404 into an LB tube, culturing under the conditions of a temperature of 28° C. and a rotation speed of 200 rpm for 2 hours, spreading on an LB plate containing 50 mg/L of rifampicin and 50 mg/L of spectinomycin until positive single clones were grown, picking out single clones for culturing and extracting the plasmids thereof, and performing enzyme digestion verification using restriction enzymes. The results showed that the structures of the recombinant expression vectors DBN100828, DBN100827, DBN100826, DBN100825, DBN100824 and DBN100823 were completely correct.

### Example 7. Acquisition and Verification of Transgenic Soybean Plants

1. Acquisition of Transgenic Soybean Plants

According to the Agrobacterium infection method conventionally used, the cotyledonary node tissue of a sterile culture of soybean variety Zhonghuang13 was co-cultured with the Agrobacterium in point 2 of Example 6, so as to introduce T-DNA (comprising the Arabidopsis thaliana Ubiquitin10 gene promoter sequence, an ALT-1-01 nucleotide sequence, an ALT-2-01 nucleotide sequence, an ALT-3-01 nucleotide sequence, the tNos terminator, the rape eukaryotic elongation factor gene 1α promoter, the Arabidopsis thaliana chloroplast transit peptide, 5-enolpyruvylshikimate-3-phosphate synthase gene and the pea RbcS gene terminator) in the recombinant expression vectors DBN100828, DBN100827, DBN100826, DBN100825, DBN100824 and DBN100823 constructed in Example 6.1 into the soybean chromosomes, obtaining soybean plants that were transformed with the recombinant expression vector DBN100828 and had an ALT-1-01 nucleotide sequence located in the cytoplasm introduced (Gm cytoplasmic ALT-1-01) and soybean plants that were transformed with the recombinant expression vector DBN100827 and had an ALT-1-01 nucleotide sequence located in the chloroplast introduced (Gm chloroplastic ALT-1-01); soybean plants that were transformed with the recombinant expression vector DBN100826 and had an ALT-2-01 nucleotide sequence located in the cytoplasm introduced (Gm cytoplasmic ALT-2-01) and soybean plants that were transformed with the recombinant expression vector DBN100825 and had an ALT-2-01 nucleotide sequence located in the chloroplast introduced (Gm chloroplastic ALT-2-01); and soybean plants that were transformed with the recombinant expression vector DBN100824 and had an ALT-3-01 nucleotide sequence located in the cytoplasm introduced (Gm cytoplasmic ALT-3-01) and soybean plants that were transformed with the recombinant expression vector DBN100823 and had an ALT-3-01 nucleotide sequence located in the chloroplast introduced (Gm chloroplastic ALT-3-01); meanwhile, wild type soybean plants were used as the control.

As regards the Agrobacterium-mediated soybean transformation, briefly, mature soybean seeds were germinated in a soybean germination culture medium (3.1 g/L of B5 salt, B5 vitamin, 20 g/L of sucrose, and 8 g/L of agar, pH 5.6), and the seeds were inoculated in a germination culture medium and cultured under the conditions of a temperature of 25±1° C.; and a photoperiod (light/dark) of 16 h/8 h. After 4-6 days of germination, soybean sterile seedlings swelled at bright green cotyledonary nodes were taken, hypocotyledonary axes were cut off 3-4 millimeters below the cotyledonary nodes, the cotyledons were cut longitudinally, and apical buds, lateral bud and seminal roots were removed. A wound was made at a cotyledonary node using the knife back of a scalpel, and the wounded cotyledonary node tissue was contacted with an Agrobacterium suspension, wherein the Agrobacterium can transfer the ALT-1-01 nucleotide sequence, the ALT-2-01 nucleotide sequence and the ALT-3-01 nucleotide sequence to the wounded cotyledonary node tissue (step 1: infection step). In this step, the cotyledonary node tissues were preferably immersed in the Agrobacterium suspension (OD₆₆₀=0.5-0.8, an infection culture medium (2.15 g/L of MS salt, B5 vitamin, 20 g/L of sucrose, 10 g/L of glucose, 40 mg/L of acetosyringone (AS), 4 g/L of 2-morpholine ethanesulfonic acid (MES), and 2 mg/L of zeatin (ZT), pH 5.3) to initiate the inoculation. The cotyledonary node tissues were co-cultured with Agrobacterium for a period of time (3 days) (step 2: co-culturing step). Preferably, the cotyledonary node tissues were cultured in a solid culture medium (4.3 g/L of MS salt, B5 vitamin, 20 g/L of sucrose, 10 g/L of glucose, 4 g/L of 2-morpholine ethanesulfonic acid (MES), 2 mg/L of zeatin, and 8 g/L of agar, pH 5.6) after the infection step. After this co-culturing stage, there can be an optional “recovery” step. In the “recovery” step, there may be at least one antibiotic (cephalosporin) known to inhibit the growth of Agrobacterium in a recovery culture medium (3.1 g/L of B5 salt, B5 vitamin, 1 g/L of 2-morpholine ethanesulfonic acid (MES), 30 g/L of sucrose, 2 mg/L of zeatin (ZT), 8 g/L of agar, 150 mg/L of cephalosporin, 100 mg/L of glutamic acid, and 100 mg/L of aspartic acid, pH 5.6), without the addition of a selective agent for a plant transformant (step 3: recovery step). Preferably, tissue blocks regenerated from the cotyledonary nodes were cultured in a solid culture medium with an antibiotic but without a selective agent, to eliminate Agrobacterium and provide a recovery stage for the infected cells. Subsequently, the tissue blocks regenerated from the cotyledonary nodes were cultured in a culture medium containing a selective agent (glyphosate), and growing transformed calli were selected (step 4: selection step). Preferably, the tissue blocks regenerated from the cotyledonary nodes were cultured in a screening solid culture medium (3.1 g/L of B5 salt, B5 vitamin, 1 g/L of 2-morpholine ethanesulfonic acid (MES), 30 g/L of sucrose, 1 mg/L of 6-benzyladenine (6-BAP), 8 g/L of agar, 150 mg/L of cephalosporin, 100 mg/L of glutamic acid, 100 mg/L of aspartic acid, and 0.25 mol/L of N-(phosphonomethyl)glycine, pH 5.6) containing a selective agent, resulting in selective growth of the transformed cells. Then, plants were regenerated from the transformed cells (step 5: regeneration step). Preferably, the tissue blocks regenerated from the cotyledonary nodes grown in a culture medium containing a selective agent were cultured in solid culture media (B5 differentiation culture medium and B5 rooting culture medium) to regenerate plants.

The screened out resistant tissues were transferred onto the B5 differentiation culture medium (3.1 g/L of B5 salt, B5 vitamin, 1 g/L of 2-morpholine ethanesulfonic acid (MES), 30 g/L of sucrose, 1 mg/L of zeatin (ZT), 8 g/L of agar, 150 mg/L of cephalosporin, 50 mg/L of glutamic acid, 50 mg/L of aspartic acid, 1 mg/L of gibberellin, 1 mg/L of auxin, and 0.25 mol/L of N-(phosphonomethyl)glycine, pH 5.6), and cultured at 25° C. for differentiation. The differentiated seedlings were transferred onto the B5 rooting culture medium (3.1 g/L of B5 salt, B5 vitamin, 1 g/L of 2-morpholine ethanesulfonic acid (MES), 30 g/L of sucrose, 8 g/L of agar, 150 mg/L of cephalosporin, and 1 mg/L of indole-3-butyric acid (IBA)), cultured in the rooting culture medium to be a height of about 10 cm at 25° C., and transferred to a glasshouse for culturing until fruiting. In the greenhouse, the plants were cultured at 26° C. for 16 hours, and then cultured at 20° C. for 8 hours every day.

2. Verification of the Transgenic Soybean Plants Using TaqMan

Leaves of about 100 mg from Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants and Gm chloroplastic ALT-3-01 soybean plants were respectively taken as samples, genomic DNAs thereof were extracted with a DNeasy Plant Maxi Kit (Qiagen), and the copy number of the EPSPS gene was detected by the Taqman probe fluorescence quantitative PCR method so as to determine the copy number of the ALT gene. At the same time, wild type soybean plants were used as controls, and detected and analyzed according to the above-mentioned method. Triple repeats were set for the experiments, and averaged.

The specific method for detecting the copy number of the EPSPS gene was as follows:

Step 11. Leaves of 100 mg from Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants, Gm chloroplastic ALT-3-01 soybean plants and wild-type soybean plants were respectively taken each, respectively ground into a homogenate in a mortar with liquid nitrogen, and 3 replicates were taken for each sample;

Step 12. Genomic DNAs of the above-mentioned samples were extracted using a DNeasy Plant Mini Kit of Qiagen, and the particular method can refer to the product manual thereof;

Step 13. The concentrations of the genomic DNAs of the above-mentioned samples were detected using NanoDrop 2000 (Thermo Scientific);

Step 14. The concentrations of the genomic DNAs of the above-mentioned samples were adjusted to a consistent concentration value which ranges from 80 to 100 ng/μL;

Step 15. The copy numbers of the samples were identified using the Taqman probe fluorescence quantitative PCR method, wherein samples for which the copy numbers had been identified and known were taken as standards, the samples of the wild type soybean plants were taken as the control, and triple repeats were taken for each sample and averaged; the sequences of fluorescence quantitative PCR primers and a probe were as follows, respectively:

the following primers and probe were used to detect the EPSPS gene sequence:

| | |
|---|---|
| primer 1: | CTGGAAGGCGAGGACGTCATCAATA, shown as |
| | SEQ ID NO: 20 in the sequence listing; |
| primer 2: | TGGCGGCATTGCCGAAATCGAG, shown as |
| | SEQ ID NO: 21 in the sequence listing; |
| primer 1: | ATGCAGGCGATGGGCGCCCGCATCCGTA, shown as |
| | SEQ ID NO: 22 in the sequence listing; |

PCR Reaction System:

| | |
|---|---|
| JumpStart ™ Tag ReadyMix ™ (Sigma) | 10 μL |
| 50 × primer/probe mixture | 1 μL |
| genomic DNA | 3 μL |
| water (ddH2O) | 6 μL |

The 50× primer/probe mixture comprises 45 μL of each primer at a concentration of 1 mM, 50 μL of the probe at a concentration of 100 μM, and 860 μL of 1×TE buffer, and was stored at 4° C. in an amber tube.

PCR Reaction Conditions:

| Step | Temperature | Time | |
|---|---|---|---|
| 21 | 95° C. | 5 | minutes |
| 22 | 95° C. | 30 | seconds |
| 23 | 60° C. | 1 | minute |

| | |
|---|---|
| 24 | back to step 22, repeated 40 times |

Data were analyzed using software SDS2. 3 (Applied Biosystems).

It was further confirmed by analyzing the experimental results of the copy number of the EPSPS gene that the ALT-1-01 nucleotide sequence, the ALT-2-01 nucleotide sequence and the ALT-3-01 nucleotide sequence had all been incorporated into the chromosomes of the detected soybean plants, and Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants and Gm chloroplastic ALT-3-01 soybean plants all resulted in single copy transgenic soybean plants.

### Example 8. Detection of Herbicide Tolerance Effects of the Transgenic Soybean Plants

1. Sulfometuron-Methyl Tolerance

The herbicide tolerance effects of Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants, Gm chloroplastic ALT-3-01 soybean plants and wild-type soybean plants on Sulfometuron-methyl were detected (at seedling stage), respectively.

Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants, Gm chloroplastic ALT-3-01 soybean plants and wild-type soybean plants were respectively taken and sprayed with Sulfometuron-methyl (120 g ai/ha, 4-fold field concentration) and a blank solvent (water). The damage degree caused by the herbicide was measured for each plant according to the leaf curl degree and the growth point damage degree 3 days after spraying (3 DAT), 7 days after spraying (7 DAT), 14 days after spraying (14 DAT) and 21 days after spraying (21 DAT), respectively: considering conditions of leaves being flat as the untreated plants and growth points being intact as having a damage degree of 0%; considering conditions of veins being locally browned, new leaves being malformed and plant growth being slow as having a damage degree of 50%; and considering conditions of veins being purple to whole plant being dead and growth points being browned and dry as having a damage degree of 100%. There were 2 strains in Gm cytoplasmic ALT-1-01 soybean plants in total (S1 and S2), 2 strains in Gm chloroplastic ALT-1-01 soybean plants in total (S3 and S4), 2 strains in Gm cytoplasmic ALT-2-01 soybean plants in total (S5 and S6), 2 strains in Gm chloroplastic ALT-2-01 soybean plants in total (S7 and S8), 2 strains in Gm cytoplasmic ALT-3-01 soybean plants in total (S9 and S10), 2 strains in Gm chloroplastic ALT-3-01 soybean plants in total (S11 and S12), and 1 strain in wild-type soybean plants (CK1) in total; and 10-15 plants were selected from each strain and tested. The results are as shown in Table 3.

TABLE 3
Experimental results of the herbicide tolerance
of transgenic soybean T₁ plants

| | | Average | Average | Average | Average |
|---|---|---|---|---|---|
| | | damage | damage | damage | damage |
| | Soybean | % | % | % | % |
| Treatment | genotypes | 3DAT | 7DAT | 14DAT | 21DAT |
| Blank | S1 | 0 | 0 | 0 | 0 |
| solvent | S2 | 0 | 0 | 0 | 0 |
| (water) | S3 | 0 | 0 | 0 | 0 |
| | S4 | 0 | 0 | 0 | 0 |
| | S5 | 0 | 0 | 0 | 0 |
| | S6 | 0 | 0 | 0 | 0 |
| | S7 | 0 | 0 | 0 | 0 |
| | S8 | 0 | 0 | 0 | 0 |
| | S9 | 0 | 0 | 0 | 0 |
| | S10 | 0 | 0 | 0 | 0 |
| | S11 | 0 | 0 | 0 | 0 |
| | S12 | 0 | 0 | 0 | 0 |
| | CK1 | 0 | 0 | 0 | 0 |
| 120 g ai/ha | S1 | 6 | 0 | 0 | 0 |
| Sulfo- | S2 | 7 | 0 | 0 | 0 |
| meturon- | S3 | 0 | 0 | 0 | 0 |
| methyl | S4 | 0 | 0 | 0 | 0 |
| (4 × Sul) | S5 | 6 | 0 | 0 | 0 |
| | S6 | 5 | 0 | 0 | 0 |
| | S7 | 0 | 0 | 0 | 0 |
| | S8 | 0 | 0 | 0 | 0 |
| | S9 | 8 | 0 | 0 | 0 |
| | S10 | 7 | 0 | 0 | 0 |
| | S11 | 0 | 0 | 0 | 0 |
| | S12 | 0 | 0 | 0 | 0 |
| | CK1 | 49 | 87 | 100 | 100 |

For soybean, 120 g ai/ha Sulfometuron-methyl herbicide is an effective dose distinguishing sensitive plants from plants having an average level of resistance. The results of Table 3 show that: the thifensulfuron hydrolase (ALT-1, ALT-2 and ALT-3) conferred a high level of Sulfometuron-methyl herbicide tolerance to the transgenic soybean plants; compared to Gm cytoplasmic ALT-1-01 soybean plants, Gm cytoplasmic ALT-2-01 soybean plants and Gm cytoplasmic ALT-3-01 soybean plants, Gm chloroplastic ALT-1-01 soybean plants, Gm chloroplastic ALT-2-01 soybean plants and Gm chloroplastic ALT-3-01 soybean plants were able to produce a higher Sulfometuron-methyl herbicide tolerance, suggesting that the thifensulfuron hydrolase (ALT-1, ALT-2 and ALT-3) gene may enhance the tolerance of soybean plants to the Sulfometuron-methyl herbicide when located in the chloroplast for expression; while the wild-type soybean plants were not tolerant to the Sulfometuron-methyl herbicide.

2. Glyphosate Tolerance

The herbicide tolerance effects of Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants, Gm chloroplastic ALT-3-01 soybean plants and wild-type soybean plants on glyphosate were detected (at seedling stage), respectively.

2 strains from Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants, Gm chloroplastic ALT-3-01 soybean plants and wild-type soybean plants were respectively taken each, and 10-15 plants were selected from each strain and tested. The plants above were sprayed with glyphosate (840 g ae/ha, 1-fold field concentration) and a blank solvent (water). The herbicide damage rate was measured for each plant according to the phytotoxicity symptoms 14 days after spraying (14 DAT): herbicide damage rate (%)=Σ(number of damaged plants at the same level×level number)/(total number of plants×highest level). Grading of the phytotoxicity symptoms is as shown in Table 5.

TABLE 5
Grading standards of the phytotoxicity degree caused by the
glyphosate herbicide to soybeans

| Phytotoxicity | |
|---|---|
| level | Symptom description |
| 1 | growing normally, without any damage symptoms |
| 2 | mild phytotoxicity, less than 10% of phytotoxicity |
| 3 | moderate phytotoxicity, able to recover later |
| 4 | relatively severe phytotoxicity, difficult to recover |
| 5 | severe phytotoxicity, unable to recover |

The results suggested that the glyphosate herbicide damage rates of Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants and Gm chloroplastic ALT-3-01 soybean plants were substantially 0%, whereas the glyphosate herbicide damage rate of wild-type soybean plants (CK1) was up to not less than 90%; thereby, Gm cytoplasmic ALT-1-01 soybean plants, Gm chloroplastic ALT-1-01 soybean plants, Gm cytoplasmic ALT-2-01 soybean plants, Gm chloroplastic ALT-2-01 soybean plants, Gm cytoplasmic ALT-3-01 soybean plants and Gm chloroplastic ALT-3-01 soybean plants were very tolerant to the glyphosate herbicide.

### Example 9. Construction of Maize Recombinant Expression Vectors

1. Construction of Maize Recombinant Cloning Vectors Containing ALT Nucleotide Sequences

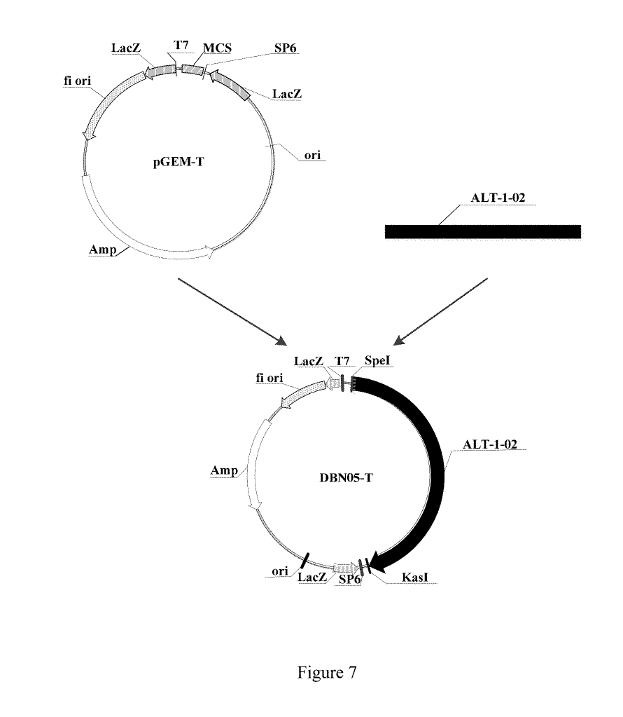

The synthetic ALT-1-02 nucleotide sequence was ligated into cloning vector pGEM-T (Promega, Madison, USA, CAT: A3600), and the operational procedure was carried out according to Promega's pGEM-T vector product instructions, obtaining a recombinant cloning vector DBN05-T, the construction process of which is as shown in FIG. 7 (wherein Amp means the ampicillin resistance gene; f1 means the origin of replication of phage f1; LacZ is LacZ initiation codon; SP6 is SP6 RNA polymerase promoter; T7 is T7 RNA polymerase promoter; ALT-1-02 is the ALT-1-02 nucleotide sequence (SEQ ID NO: 3); and MCS is a multiple cloning site).

According to the method in point 1 of Example 2, Escherichia coli T1 competent cells were transformed with the recombinant cloning vector DBN05-T using the heat shock method, and the plasmids in the cells were extracted through the alkaline method. After identifying the extracted plasmid by SpeI and KasI digestion, positive clones were verified by sequencing. The results showed that the inserted ALT-1-02 nucleotide sequence in the recombinant cloning vector DBN05-T was the nucleotide sequence shown as SEQ ID NO: 3 in the sequence listing, that is, the ALT-1-02 nucleotide sequence was inserted correctly.

According to the above-mentioned method for constructing the recombinant cloning vector DBN05-T, the synthetic ALT-2-02 nucleotide sequence was ligated into a cloning vector pGEM-T, obtaining a recombinant cloning vector DBN06-T, wherein ALT-2-02 was the ALT-2-02 nucleotide sequence (SEQ ID NO: 6). Enzyme digestion and sequencing verified that the ALT-2-02 nucleotide sequence was correctly inserted into the recombinant cloning vector DBN06-T.

According to the above-mentioned method for constructing the recombinant cloning vector DBN05-T, the synthetic ALT-3-02 nucleotide sequence was ligated into a cloning vector pGEM-T, obtaining a recombinant cloning vector DBN07-T, wherein ALT-3-02 was the ALT-3-02 nucleotide sequence (SEQ ID NO: 9). Enzyme digestion and sequencing verified that the ALT-3-02 nucleotide sequence was correctly inserted into the recombinant cloning vector DBN07-T.

2. Construction of Maize Recombinant Expression Vectors Containing ALT Nucleotide Sequences

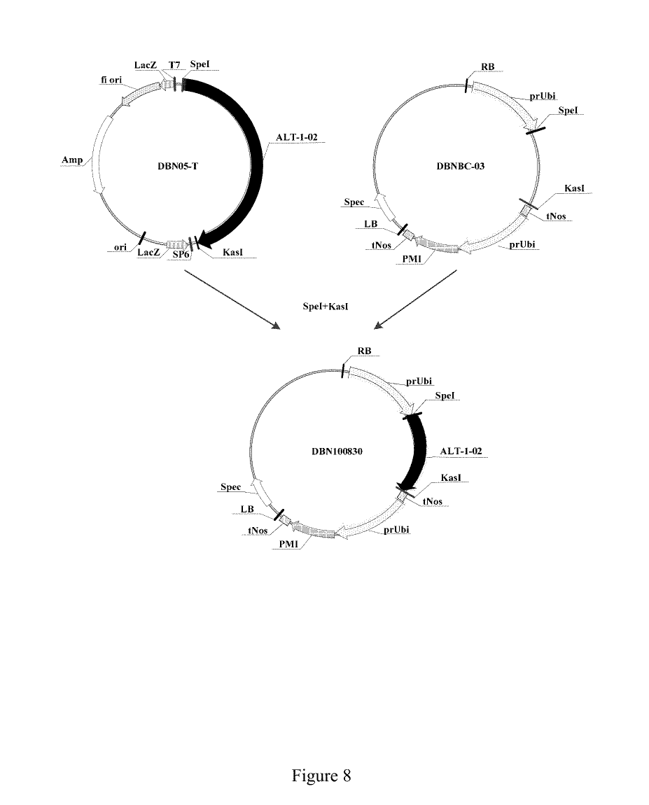

The recombinant cloning vector DBN05-T and an expression vector DBNBC-03 (vector backbone: pCAMBIA2301 (which can be provided by the CAMBIA institution)) were digested with restriction enzymes SpeI and KasI, respectively; the excised ALT-1-02 nucleotide sequence fragment was inserted between the SpeI and KasI sites in the expression vector DBNBC-03; and it is well known to a person skilled in the art to construct a vector using conventional enzyme digestion methods, constructing a recombinant expression vector DBN100830 (located in the cytoplasm), the construction process of which is as shown FIG. 8 (Spec: the spectinomycin gene; RB: the right boundary; prUbi: the maize Ubiquitin 1 gene promoter (SEQ ID NO: 23); ALT-1-02: the ALT-1-02 nucleotide sequence (SEQ ID NO: 3); tNos: the terminator of nopaline synthase gene (SEQ ID NO:13); PMI: the phosphomannose isomerase gene (SEQ ID NO: 24); LB: the left boundary).

According to the method in point 2 of Example 2, Escherichia coli T1 competent cells were transformed with the recombinant expression vector DBN100830 using the heat shock method, and the plasmids in the cells were extracted through the alkaline method. The extracted plasmid was identified after digesting with restriction enzymes SpeI and KasI, and positive clones were identified by sequencing. The results showed that the nucleotide sequence between the SpeI and KasI sites in the recombinant expression vector DBN100830 was the nucleotide sequence shown as SEQ ID NO: 3 in the sequence listing, i.e., the ALT-1-02 nucleotide sequence.

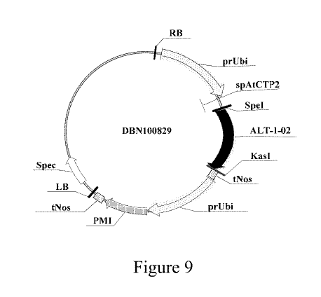

According to the above-mentioned method for constructing the recombinant expression vector DBN100830, a recombinant expression vector DBN100829 (located in the chloroplast) containing the ALT-1-02 nucleotide sequence was constructed, the vector structure of which is as shown in FIG. 9 (vector backbone: pCAMBIA2301 (which can be provided by the CAMBIA institution); Spec: the spectinomycin gene; RB: the right boundary; prUbi: the maize Ubiquitin 1 gene promoter (SEQ ID NO: 23); spAtCTP2: the Arabidopsis thaliana chloroplast transit peptide (SEQ ID NO: 17); ALT-1-02: the ALT-1-02 nucleotide sequence (SEQ ID NO: 3); tNos: the terminator of nopaline synthase gene (SEQ ID NO:13); PMI: the phosphomannose isomerase gene (SEQ ID NO: 24); LB: the left boundary). Positive clones were verified by sequencing. The results showed that the inserted ALT-1-02 nucleotide sequence in the recombinant expression vector DBN100829 was the nucleotide sequence shown as SEQ ID NO: 3 in the sequence listing, that is, the ALT-1-02 nucleotide sequence was inserted correctly.

According to the above-mentioned method for constructing the recombinant expression vector DBN100830, the ALT-2-02 nucleotide sequence excised by SpeI and KasI from digested recombinant cloning vector DBN06-T was inserted into the expression vector DBNBC-03, obtaining a recombinant expression vector DBN100832. Enzyme digestion and sequencing verified that the nucleotide sequence in the recombinant expression vector DBN100832 contained the nucleotide sequence shown as SEQ ID NO: 6 in the sequence listing, that is, the ALT-2-02 nucleotide sequence was inserted correctly.

According to the above-mentioned method for constructing the recombinant expression vector DBN100829, the ALT-2-02 nucleotide sequence excised by SpeI and KasI from digested recombinant cloning vector DBN06-T was inserted into the expression vector DBNBC-03, obtaining a recombinant expression vector DBN100831 (containing spAtCTP2, located in the chloroplast). Enzyme digestion and sequencing verified that the nucleotide sequence in the recombinant expression vector DBN100831 contained the nucleotide sequence shown as SEQ ID NO: 6 in the sequence listing, that is, the ALT-2-02 nucleotide sequence was inserted correctly.

According to the above-mentioned method for constructing the recombinant expression vector DBN100830, the ALT-3-02 nucleotide sequence excised by SpeI and KasI from digested recombinant cloning vector DBN07-T was inserted into the expression vector DBNBC-03, obtaining a recombinant expression vector DBN100834. Enzyme digestion and sequencing verified that the nucleotide sequence in the recombinant expression vector DBN100834 contained the nucleotide sequence shown as SEQ ID NO: 9 in the sequence listing, that is, the ALT-3-02 nucleotide sequence was inserted correctly.

According to the above-mentioned method for constructing the recombinant expression vector DBN100829, the ALT-3-02 nucleotide sequence excised by SpeI and KasI from digested recombinant cloning vector DBN07-T was inserted into the expression vector DBNBC-03, obtaining a recombinant expression vector DBN100833 (containing spAtCTP2, located in the chloroplast). Enzyme digestion and sequencing verified that the nucleotide sequence in the recombinant expression vector DBN100833 contained the nucleotide sequence shown as SEQ ID NO: 9 in the sequence listing, that is, the ALT-3-02 nucleotide sequence was inserted correctly.

3. Transformation of Agrobacterium with the Maize Recombinant Expression Vectors

Agrobacterium LBA4404 (Invitrogen, Chicago, USA, CAT: 18313-015) was transformed with the recombinant expression vectors DBN100830, DBN100829, DBN100832, DBN100831, DBN100834 and DBN100833 which had been correctly constructed using a liquid nitrogen method, with the following transformation conditions: placing 100 μL of Agrobacterium LBA4404, and 3 μL of plasmid DNA (recombinant expression vector) in liquid nitrogen for 10 minutes, warm water bathing at 37° C. for 10 minutes; inoculating the transformed Agrobacterium LBA4404 into an LB tube, culturing under the conditions of a temperature of 28° C. and a rotation speed of 200 rpm for 2 hours, spreading on an LB plate containing 50 mg/L of rifampicin and 50 mg/L of spectinomycin until positive single clones were grown, picking out single clones for culturing and extracting the plasmids thereof, and performing enzyme digestion verification using restriction enzymes. The results showed that the structures of the recombinant expression vectors DBN100830, DBN100829, DBN100832, DBN100831, DBN100834 and DBN100833 were completely correct.

### Example 10. Acquisition and Verification of Transgenic Maize Plants

According to the Agrobacterium infection method conventionally used, young embryos of a sterile culture of maize variety Zong31 (Z31) were co-cultured with the Agrobacterium in point 3 of Example 9, so as to introduce T-DNA (comprising the maize Ubiquitin) gene promoter sequence, an ALT-1-02 nucleotide sequence, an ALT-2-02 nucleotide sequence, an ALT-3-02 nucleotide sequence, the Arabidopsis thaliana chloroplast transit peptide, the PMI gene and the tNos terminator sequence) in the recombinant expression vectors DBN100830, DBN100829, DBN100832, DBN100831, DBN100834 and DBN100833 constructed in point 2 of Example 9 into the maize chromosomes, obtaining maize plants that were transformed with the recombinant expression vector DBN100830 and had an ALT-1-02 nucleotide sequence located in the cytoplasm introduced (Zm cytoplasmic ALT-1-02) and maize plants that were transformed with the recombinant expression vector DBN100829 and had an ALT-1-02 nucleotide sequence located in the chloroplast introduced (Zm chloroplastic ALT-1-02); maize plants that were transformed with the recombinant expression vector DBN100832 and had an ALT-2-02 nucleotide sequence located in the cytoplasm introduced (Zm cytoplasmic ALT-2-02) and maize plants that were transformed with the recombinant expression vector DBN100831 and had an ALT-2-02 nucleotide sequence located in the chloroplast introduced (Zm chloroplastic ALT-2-02); maize plants that were transformed with the recombinant expression vector DBN100834 and had an ALT-3-02 nucleotide sequence located in the cytoplasm introduced (Zm cytoplasmic ALT-3-02) and maize plants that were transformed with the recombinant expression vector DBN100833 and had an ALT-3-02 nucleotide sequence located in the chloroplast introduced (Zm chloroplastic ALT-3-02); meanwhile, wild type maize plants were used as the control.

For the Agrobacterium-mediated maize transformation, briefly, immature young embryos were separated from maize, and contacted with an Agrobacterium suspension, wherein the Agrobacterium can transfer the ALT-1-02 nucleotide sequence, the ALT-2-02 nucleotide sequence and the ALT-3-02 nucleotide sequence to at least one cell of one of young embryos (step 1: infection step). In this step, the young embryos were preferably immersed in an Agrobacterium suspension (OD₆₆₀=0.4-0.6, an infection culture medium (4.3 g/L of MS salt, MS vitamin, 300 mg/L of casein, 68.5 g/L of sucrose, 36 g/L of glucose, 40 mg/L of acetosyringone (AS), and 1 mg/L of 2,4-dichlorphenoxyacetic acid (2,4-D), pH 5.3) to initiate the inoculation. The young embryos were co-cultured with Agrobacterium for a period of time (3 days) (step 2: co-culturing step). Preferably, the young embryos were cultured in a solid culture medium (4.3 g/L of MS salt, MS vitamin, 300 mg/L of casein, 20 g/L of sucrose, 10 g/L of glucose, 100 mg/L of acetosyringone (AS), 1 mg/L of 2,4-dichlorphenoxyacetic acid (2,4-D), and 8 g/L of agar, pH 5.8) after the infection step. After this co-culturing stage, there can be an optional “recovery” step. In the “recovery” step, there may be at least one antibiotic (cephalosporin) known to inhibit the growth of Agrobacterium in a recovery culture medium (4.3 g/L of MS salt, MS vitamin, 300 mg/L of casein, 30 g/L of sucrose, 1 mg/L of 2,4-dichlorphenoxyacetic acid (2,4-D), and 3 g/L of phytagel, pH 5.8), without the addition of a selective agent for a plant transformant (step 3: recovery step). Preferably, the young embryos were cultured in a solid culture medium with an antibiotic but without a selective agent, to eliminate Agrobacterium and provide a recovery stage for the infected cells. Subsequently, the inoculated young embryos were cultured in a culture medium containing a selective agent (mannose), and growing transformed calli were selected (step 4: selection step). Preferably, the young embryos were cultured in a screening solid culture medium (4.3 g/L of MS salt, MS vitamin, 300 mg/L of casein, 30 g/L of sucrose, 12.5 g/L of mannose, 1 mg/L of 2,4-dichlorphenoxyacetic acid (2,4-D), and 3 g/L of phytagel, pH 5.8) with a selective agent, resulting in selective growth of transformed cells. Then, plants were regenerated from the calli (step 5: regeneration step). Preferably, the calli grown in a culture medium containing a selective agent were cultured in solid culture media (MS differentiation culture medium and MS rooting culture medium) to regenerate plants.

Resistant calli screened out were transferred onto the MS differentiation culture medium (4.3 g/L of MS salt, MS vitamin, 300 mg/L of casein, 30 g/L of sucrose, 2 mg/L of 6-benzyladenine, 5 g/L of mannose, and 3 g/L of phytagel, pH 5.8), and cultured at 25° C. for differentiation. The differentiated seedlings were transferred onto the MS rooting culture medium (2.15 g/L of MS salt, MS vitamin, 300 mg/L of casein, 30 g/L of sucrose, 1 mg/L of indole-3-acetic acid, and 3 g/L of phytagel, pH 5.8), cultured at 25° C. to a height of about 10 cm, and transferred to a glasshouse for culturing until fruiting. In the greenhouse, the plants were cultured at 28° C. for 16 hours, and then cultured at 20° C. for 8 hours every day.

2. Verification of the Transgenic Maize Plants Using TaqMan

According to the method in point 2 of Example 7 for verifying the transgenic soybean plants using TaqMan, Zm cytoplasmic ALT-1-02 maize plants, Zm chloroplastic ALT-1-02 maize plants, Zm cytoplasmic ALT-2-02 maize plants, Zm chloroplastic ALT-2-02 maize plants, Zm cytoplasmic ALT-3-02 maize plants and Zm chloroplastic ALT-3-02 maize plants were detected and analyzed. The copy number of the PMI gene was detected by the Taqman probe fluorescence quantitative PCR method so as to determine the copy number of the ALT gene. Meanwhile, wild type maize plants were used as the control, and detected and analyzed according to the above-mentioned method. Triple repeats were set for the experiments, and averaged.

The following primers and probe were used to detect the PMI gene sequence:

| | |
|---|---|
| primer 3: | GCTGTAAGAGCTTACTGAAAAAATTAACA, shown as |
| | SEQ ID NO: 25 in the sequence listing |
| primer 4: | CGATCTGCAGGTCGACGG, shown as SEQ ID NO: |
| | 26 in the sequence listing; |
| primer 2: | TCTCTTGCTAAGCTGGGAGCTCGATCC, shown as |
| | SEQ ID NO: 27 in the sequence listing. |

It was further confirmed by analyzing the experimental results of the copy number of the PMI gene that the ALT-1-02 nucleotide sequence, the ALT-2-02 nucleotide sequence and the ALT-3-02 nucleotide sequence had all been incorporated into the chromosomes of the detected maize plants, and Zm cytoplasmic ALT-1-02 maize plants, Zm chloroplastic ALT-1-02 maize plants, Zm cytoplasmic ALT-2-02 maize plants, Zm chloroplastic ALT-2-02 maize plants, Zm cytoplasmic ALT-3-02 maize plants and Zm chloroplastic ALT-3-02 maize plants all resulted in single copy transgenic maize plants.

### Example 11. Detection of Herbicide Tolerance Effects of the Transgenic Maize Plants

The herbicide tolerance effects of Zm cytoplasmic ALT-1-02 maize plants, Zm chloroplastic ALT-1-02 maize plants, Zm cytoplasmic ALT-2-02 maize plants, Zm chloroplastic ALT-2-02 maize plants, Zm cytoplasmic ALT-3-02 maize plants, Zm chloroplastic ALT-3-02 maize plants and wild-type maize plants on Sulfometuron-methyl were detected (at V3-V4 stage), respectively.

Zm cytoplasmic ALT-1-02 maize plants, Zm chloroplastic ALT-1-02 maize plants, Zm cytoplasmic ALT-2-02 maize plants, Zm chloroplastic ALT-2-02 maize plants, Zm cytoplasmic ALT-3-02 maize plants, Zm chloroplastic ALT-3-02 maize plants and wild-type maize plants were respectively taken and sprayed with Sulfometuron-methyl (120 g ai/ha, 4-fold field concentration) and a blank solvent (water). The damage degree caused by the herbicide was measured for each plant according to the plant growth status 3 days after spraying (3 DAT), 7 days after spraying (7 DAT), 14 days after spraying (14 DAT) and 21 days after spraying (21 DAT), respectively: considering a growth status equivalent to that of the untreated plants as having a damage degree of 0%; considering conditions of leaves being partially chlorotic and yellow but substantially not affecting the plant normal growth as having a damage degree of 50%; and considering the whole plant being purple and dying as having a damage degree of 100%. There were 2 strains in Zm cytoplasmic ALT-1-02 maize plants in total (S13 and S14), 2 strains in Zm chloroplastic ALT-1-02 maize plants in total (S15 and S16), 2 strains in Zm cytoplasmic ALT-2-02 maize plants in total (S17 and S18), 2 strains in Zm chloroplastic ALT-2-02 maize plants in total (S19 and S20), 2 strains in Zm cytoplasmic ALT-3-02 maize plants in total (S21 and S22), 2 strains in Zm chloroplastic ALT-3-02 maize plants in total (S23 and S24), and 1 strain in wild-type maize plants (CK2) in total; and 10-15 plants were selected from each strain and tested. The results are as shown in Table 4.

TABLE 4
Experimental results of the herbicide tolerance
of transgenic maize T₁ plants

| | | Average | Average | Average | Average |
|---|---|---|---|---|---|
| | | damage | damage | damage | damage |
| | Maize | % | % | % | % |
| Treatment | genotypes | 3DAT | 7DAT | 14DAT | 21DAT |
| Blank | S13 | 0 | 0 | 0 | 0 |
| solvent | S14 | 0 | 0 | 0 | 0 |
| (water) | S15 | 0 | 0 | 0 | 0 |
| | S16 | 0 | 0 | 0 | 0 |
| | S17 | 0 | 0 | 0 | 0 |
| | S18 | 0 | 0 | 0 | 0 |
| | S19 | 0 | 0 | 0 | 0 |
| | S20 | 0 | 0 | 0 | 0 |
| | S21 | 0 | 0 | 0 | 0 |
| | S22 | 0 | 0 | 0 | 0 |
| | S23 | 0 | 0 | 0 | 0 |
| | S24 | 0 | 0 | 0 | 0 |
| | CK2 | 0 | 0 | 0 | 0 |
| 120 g ai/ha | S13 | 6 | 0 | 0 | 0 |
| Sulfo- | S14 | 7 | 0 | 0 | 0 |
| meturon- | S15 | 0 | 0 | 0 | 0 |
| methyl | S16 | 0 | 0 | 0 | 0 |
| (4× Sul.) | S17 | 6 | 0 | 0 | 0 |
| | S18 | 5 | 0 | 0 | 0 |
| | S19 | 0 | 0 | 0 | 0 |
| | S20 | 0 | 0 | 0 | 0 |
| | S21 | 5 | 0 | 0 | 0 |
| | S22 | 5 | 0 | 0 | 0 |
| | S23 | 0 | 0 | 0 | 0 |
| | S24 | 0 | 0 | 0 | 0 |
| | CK2 | 44 | 87 | 100 | 100 |

For maize, 120 g ai/ha Sulfometuron-methyl herbicide is an effective dose distinguishing sensitive plants from plants having an average level of resistance. The results of Table 4 show that: the thifensulfuron hydrolase (ALT-1, ALT-2 and ALT-3) conferred a high level of Sulfometuron-methyl herbicide tolerance on the transgenic maize plants; compared to Zm cytoplasmic ALT-1-02 maize plants, Zm cytoplasmic ALT-2-02 maize plants and Zm cytoplasmic ALT-3-02 maize plants, Zm chloroplastic ALT-1-02 maize plants, Zm chloroplastic ALT-2-02 maize plants and Zm chloroplastic ALT-3-02 maize plants were able to produce a higher Sulfometuron-methyl herbicide tolerance, suggesting that the thifensulfuron hydrolase (ALT-1, ALT-2 and ALT-3) gene may enhance the tolerance of maize plants to the Sulfometuron-methyl herbicide when located in the chloroplast for expression; while the wild-type maize plants were not tolerant to the Sulfometuron-methyl herbicide.

In summary, the present invention discloses for the first time that a thifensulfuron hydrolase (ALT-1, ALT-2 and ALT-3) can show a high tolerance to a Sulfometuron-methyl herbicide, Arabidopsis thaliana plants, soybean plants and maize plants containing nucleotide sequences encoding the thifensulfuron hydrolase are strongly tolerant to the Sulfometuron-methyl herbicide and can at least tolerate 1-fold field concentration, and thus the hydrolase has broad application prospects in plants.

Finally, it should be stated that the above embodiments are merely used for illustrating rather than limiting the technical solution of the present invention; and although the present invention has been described in detail with reference to the preferred embodiments, a person skilled in the art should understand that modifications or equivalent substitutions may be made to the technical solution of the present invention without departing from the spirit and scope of the technical solution of the present invention.

1. A method for controlling weeds, wherein the method comprises applying a herbicide containing an effective dose of Sulfometuron-methyl to a plant growth environment where at least one transgenic plant is present, wherein the transgenic plant comprises a nucleotide sequence encoding a thifensulfuron hydrolase in its genome, and compared to other plants without the nucleotide sequence encoding the thifensulfuron hydrolase, the transgenic plant has reduced plant damage and/or an increased plant yield.

2. (canceled)

3. (canceled)

4. The method for controlling weeds according to claim 1, wherein the transgenic plant is maize, soybean, Arabidopsis thaliana, cotton, rape, rice, sorghum, wheat, barley, millet, sugar cane or oat.

5. The method for controlling weeds according to claim 1, wherein the amino acid sequence of the thifensulfuron hydrolase comprises an amino acid sequence as shown in SEQ ID NO: 1, SEQ ID NO: 4 or SEQ ID NO: 7.

6. The method for controlling weeds according to claim 5, wherein the nucleotide sequence of the thifensulfuron hydrolase comprises:
(a) a nucleotide sequence encoding the amino acid sequence as shown in SEQ ID NO: 1, SEQ ID NO: 4 or SEQ ID NO: 7; or
(b) a nucleotide sequence as shown in SEQ ID NO: 2 or SEQ ID NO: 3; or
(c) a nucleotide sequence as shown in SEQ ID NO: 5 or SEQ ID NO: 6; or
(d) a nucleotide sequence as shown in SEQ ID NO: 8 or SEQ ID NO: 9.

7. The method for controlling weeds according to claim 1, wherein the transgenic plant may also comprise at least one second nucleotide different from the nucleotide sequence encoding the thifensulfuron hydrolase.

8. The method for controlling weeds according to claim 7, wherein the second nucleotide encodes a selectable marker protein, a protein with a synthetic activity, a protein with a decomposing activity, an anti-biostress protein, an anti-nonbiostress protein, a male sterile protein, a protein affecting a plant yield and/or a protein affecting plant quality.

9. The method for controlling weeds according to claim 8, wherein the second nucleotide encodes 5-enolpyruvylshikimate-3-phosphate synthase, glyphosate oxidoreductase, glyphosate-N-acetyltransferase, glyphosate decarboxylase, glufosinate acetyltransferase, α-ketoglutarate-dependent dioxygenase, dicamba monooxygenase, 4-hydroxyphenylpyruvate dioxygenase, acetolactate synthase, cytochrome-like proteins and/or protoporphyrinogen oxidase.

10. The method for controlling weeds according to claim 1, wherein the herbicide containing an effective dose of Sulfometuron-methyl also includes glyphosate herbicides, glufosinate herbicides, auxin herbicides, graminicides, pre-emergence selective herbicides and/or post-emergence selective herbicides.

11. The method for controlling weeds according to claim 1, the weeds are glyphosate-tolerant weeds, wherein the method comprises applying effective doses of a Sulfometuron-methyl herbicide and a glyphosate herbicide to a field where at least one transgenic plant is planted, wherein the field includes glyphosate-tolerant weeds or seeds thereof, the transgenic plant comprises a nucleotide sequence encoding a thifensulfuron hydrolase and a nucleotide sequence encoding a glyphosate-tolerant protein in its genome, and compared to other plants without the nucleotide sequence encoding the thifensulfuron hydrolase and/or the nucleotide sequence encoding the glyphosate-tolerant protein, the transgenic plant has reduced plant damage and/or an increased plant yield.

12. (canceled)

13. (canceled)

14. (canceled)

15. (canceled)

16. (canceled)

17. (canceled)

18. The method for controlling weeds according to claim 11, wherein the glyphosate-tolerant protein includes 5-enolpyruvylshikimate-3-phosphate synthase, glyphosate oxidoreductase, glyphosate-N-acetyltransferase or glyphosate decarboxylase.

19. The method for controlling weeds according to claim 18, wherein the amino acid sequence of the glyphosate-tolerant protein comprises an amino acid sequence as shown in SEQ ID NO: 10.

20. The method for controlling weeds according to claim 19, wherein the nucleotide sequence of the glyphosate-tolerant protein comprises:
(a) a nucleotide sequence encoding the amino acid sequence as shown in SEQ ID NO: 10; or
(b) a nucleotide sequence as shown in SEQ ID NO: 11.

21. A planting system for controlling weeds growth, wherein the planting system comprises a Sulfometuron-methyl herbicide and a plant growth environment where at least one transgenic plant is present, and a herbicide containing an effective dose of Sulfometuron-methyl is applied to the plant growth environment where at least one transgenic plant is present, wherein the transgenic plant comprises a nucleotide sequence encoding a thifensulfuron hydrolase in its genome, and compared to other plants without the nucleotide sequence encoding the thifensulfuron hydrolase, the transgenic plant has reduced plant damage and/or an increased plant yield.

22. (canceled)

23. (canceled)

24. The planting system for controlling weeds growth according to claim 21, wherein the transgenic plant is maize, soybean, Arabidopsis thaliana, cotton, rape, rice, sorghum, wheat, barley, millet, sugar cane or oat.

25. The planting system for controlling weeds growth according to claim 21, wherein the amino acid sequence of the thifensulfuron hydrolase comprises an amino acid sequence as shown in SEQ ID NO: 1, SEQ ID NO: 4 or SEQ ID NO: 7.

26. The planting system for controlling weeds growth according to claim 25, wherein the nucleotide sequence of the thifensulfuron hydrolase comprises:
(a) a nucleotide sequence encoding the amino acid sequence as shown in SEQ ID NO: 1, SEQ ID NO: 4 or SEQ ID NO: 7; or
(b) a nucleotide sequence as shown in SEQ ID NO: 2 or SEQ ID NO: 3; or
(c) a nucleotide sequence as shown in SEQ ID NO: 5 or SEQ ID NO: 6; or
(d) a nucleotide sequence as shown in SEQ ID NO: 8 or SEQ ID NO: 9.

27. The planting system for controlling weeds growth according to claim 21, wherein the transgenic plant may also comprise at least one second nucleotide different from the nucleotide sequence encoding the thifensulfuron hydrolase.

28. The planting system for controlling weeds growth according to claim 27, wherein the second nucleotide encodes a selectable marker protein, a protein with a synthetic activity, a protein with a decomposing activity, an anti-biostress protein, an anti-nonbiostress protein, a male sterile protein, a protein affecting a plant yield and/or a protein affecting plant quality.

29. The planting system for controlling weeds growth according to claim 28, wherein the second nucleotide encodes 5-enolpyruvylshikimate-3-phosphate synthase, glyphosate oxidoreductase, glyphosate-N-acetyltransferase, glyphosate decarboxylase, glufosinate acetyltransferase, α-ketoglutarate-dependent dioxygenase, 4-hydroxyphenylpyruvate dioxygenase, acetolactate synthase, cytochrome-like proteins and/or protoporphyrinogen oxidase.

30. The planting system for controlling weeds growth according to claim 21, wherein the herbicide containing a herbicidally effective dose of Sulfometuron-methyl also includes glyphosate herbicides, glufosinate herbicides, auxin herbicides, graminicides, pre-emergence selective herbicides and/or post-emergence selective herbicides.

31. The planting system for controlling weeds growth according to claim 21, the weeds are glyphosate-tolerant weeds, wherein the planting system comprises a Sulfometuron-methyl herbicide, a glyphosate herbicide and a field where at least one transgenic plant is planted, and effective doses of the Sulfometuron-methyl herbicide and the glyphosate herbicide are applied to the field where at least one transgenic plant is planted, wherein the field includes glyphosate-tolerant weeds or seeds thereof, the transgenic plant comprises a nucleotide sequence encoding a thifensulfuron hydrolase and a nucleotide sequence encoding a glyphosate-tolerant protein in its genome, and compared to other plants without the nucleotide sequence encoding the thifensulfuron hydrolase and/or the nucleotide sequence encoding the glyphosate-tolerant protein, the transgenic plant has reduced plant damage and/or an increased plant yield.

32. (canceled)

33. (canceled)

34. (canceled)

35. (canceled)

36. (canceled)

37. (canceled)

38. The planting system for controlling weeds growth according to claim 31, wherein the glyphosate-tolerant protein includes 5-enolpyruvylshikimate-3-phosphate synthase, glyphosate oxidoreductase, glyphosate-N-acetyltransferase or glyphosate decarboxylase.

39. The planting system for controlling weeds growth according to claim 38, wherein the amino acid sequence of the glyphosate-tolerant protein comprises an amino acid sequence as shown in SEQ ID NO: 10.

40. The planting system for controlling weeds growth according to claim 39, wherein the nucleotide sequence of the glyphosate-tolerant protein comprises:
(a) a nucleotide sequence encoding the amino acid sequence as shown in SEQ ID NO: 10; or
(b) a nucleotide sequence as shown in SEQ ID NO: 11.

41. (canceled)

42. (canceled)

43. (canceled)

44. A method for degrading a Sulfometuron-methyl herbicide with a thifensulfuron hydrolase, wherein the method comprises applying a herbicide containing an effective dose of Sulfometuron-methyl to a plant growth environment where at least one transgenic plant is present, wherein the transgenic plant comprises a nucleotide sequence encoding the thifensulfuron hydrolase in its genome, and compared to other plants without the nucleotide sequence encoding the thifensulfuron hydrolase, the transgenic plant has reduced plant damage and/or an increased plant yield.

45. (canceled)

46. (canceled)

47. (canceled)

48. (canceled)


SEQUENCE LISTING (27 sequences)

SEQ ID NO: 1 - protein, 398 aa, Methylophilus sp.
METDKKTGTSRRSFVKAAGTGAIGIATLPLSTATAFAETDNVELAQSKRKVVLAEQGSFYIGGRTVTGPGKFDPSKPVIPYSNEGATFYINQMYVNFQAPVRPRGLPLVFWHGGGLTGHIWESTPDGRPGFQTLFVQDRHTVYTIDQPGRGRGNIPTFNGPFGQLEEESIVNTVTGNSSKEGAWVRDRLGPAPGQFFENSQFPRGYEDNYFKEMGFSPSISSDEIVDAVVKLVTHIGPCVLVTHSASGVLGMRVATHAKNVRGIVAYEPATSIFPKGKVPEIPPLADKKSQIFPPFEIQESYFKKLAKIPIQFVFGDNIPKNPKSAYWFLDWWRVTRYAHSLSLEAINKLGGQASLLDLPTAGLRGNTHFPFTDRNNVQVASLLSDFLGKHGLDQNES

SEQ ID NO: 2 - DNA, 1197 bp, Artificial Sequence
  ALT-1-01 Nucleotide sequence
atggagactgataagaaaactggcacatctagaaggtcatttgttaaggctgcaggaacaggtgccattggaatcgctacccttccattgtccaccgccactgctttcgcagagactgataacgtggaactcgcccaatctaagagaaaggtggtgctggctgaacaagggtcattttacatagggggtaggactgttactggtcctggcaagtttgatccatccaaacctgtgataccctacagtaacgaaggagcaacattctatattaaccaaatgtatgttaacttccaggccccagtgagacctaggggacttccattggttttctggcatggaggtggcttgactggtcacatctgggagtctacacctgacggcagacccgggtttcaaaccctcttcgttcaggataggcataccgtgtacactattgaccaacctgggagaggaaggggtaacatcccaacttttaacggacctttcggacagttggaggaagagagtattgttaacactgtgacaggaaattcttcaaaggaaggtgcctgggtgagagataggcttggccctgctcccgggcaatttttcgagaactctcagtttcctagaggctatgaagacaattactttaaggagatgggattcagcccatctatatccagtgatgaaattgttgacgctgttgtgaaactcgtgacccatattggtccttgtgttctggtgactcactcagcatccggcgttcttgggatgagagtggctacacacgcaaagaatgttaggggaattgtggcctatgaaccagctacctcaatcttccccaagggaaaagttccagagataccacctctcgctgataagaaaagccaaatctttcccccattcgaaatacaggagtcttactttaagaaacttgccaagattccaatccaatttgttttcggagataacatccccaagaatccaaaatcagcatattggttcctggactggtggagagtgacaagatacgcacatagtctcagcctggaggccataaacaaattggggggacaagcttcccttttggatcttcctactgcaggattgagaggtaatacacactttcccttcaccgataggaacaatgttcaggtggcttctctcctgtcagactttctgggtaaacacggtctggatcaaaatgagagctga

SEQ ID NO: 3 - DNA, 1197 bp, Artificial Sequence
  ALT-1-02 Nucleotide sequence
atggagacagacaagaagaccggcacgtcccgcaggagcttcgtgaaggctgctggcaccggggctatcgggattgctacgctcccactgtcgacagctactgccttcgcggagactgataacgtggagctggcgcagagcaagaggaaggtggttctggctgagcaggggtcgttctacattggggggcggactgtgaccgggcccggcaagttcgacccatcgaagcctgtcattccgtactctaacgagggcgctacgttctacatcaaccagatgtacgtgaatttccaggctcccgtccgcccaaggggcctcccactggtgttctggcacggcgggggcctgacaggccatatctgggagtccactccagatggccgcccagggttccagacactcttcgttcaggacaggcacacagtgtacactattgatcagccagggaggggcagggggaacatccctaccttcaatggcccattcgggcagctggaggaggagtccatcgtgaacaccgtcacgggcaattccagcaaggagggggcttgggtcagggaccggctcggcccggccccagggcagttcttcgagaactctcagttcccccggggctacgaggataattacttcaaggagatgggcttctcaccatccatctcgtctgacgagattgtcgatgccgtggtcaagctcgttacccacatcggcccttgcgttctggtgacgcatagcgcttcgggcgtcctcgggatgagggttgctacacatgcgaagaacgttcgcggcatcgtggcttacgagccggccacttccattttccccaagggcaaggtgccagagatcccaccactggccgacaagaagtcacagatcttcccacctttcgagattcaggagtcctacttcaagaagctcgctaagatccccattcagttcgtgttcggcgacaacattcctaagaatccgaagagcgcgtactggttcctggattggtggcgcgtcacgcgctacgcgcactctctctcactggaggctatcaacaagctcgggggccaggcctcgctcctggacctccctaccgctggcctgagggggaacacccatttcccgttcacggatcggaacaatgtccaggttgcgtccctcctgagcgatttcctcggcaagcacgggctggatcagaatgagtcttga

SEQ ID NO: 4 - protein, 369 aa, Artificial Sequence
  ALT-2 Amino acid sequence
METDNVELAQSKRKVVLAEQGSFYIGGRTVTGPGKFDPSKPVIPYSNEGATFYINQMYVNFQAPVRPRGLPLVFWHGGGLTGHIWESTPDGRPGFQTLFVQDRHTVYTIDQPGRGRGNIPTFNGPFGQLEEESIVNTVTGNSSKEGAWVRDRLGPAPGQFFENSQFPRGYEDNYFKEMGFSPSISSDEIVDAVVKLVTHIGPCVLVTHSASGVLGMRVATHAKNVRGIVAYEPATSIFPKGKVPEIPPLADKKSQIFPPFEIQESYFKKLAKIPIQFVFGDNIPKNPKSAYWFLDWWRVTRYAHSLSLEAINKLGGQASLLDLPTAGLRGNTHFPFTDRNNVQVASLLSDFLGKHGLDQNESKLAAALE

SEQ ID NO: 5 - DNA, 1110 bp, Artificial Sequence
  ALT-2-01 Nucleotide sequence
atggagactgataacgtggaactcgcccaatctaagagaaaggtggtgctggctgaacaagggtcattttacatagggggtaggactgttactggtcctggcaagtttgatccatccaaacctgtgataccctacagtaacgaaggagcaacattctatattaaccaaatgtatgttaacttccaggccccagtgagacctaggggacttccattggttttctggcatggaggtggcttgactggtcacatctgggagtctacacctgacggcagacccgggtttcaaaccctcttcgttcaggataggcataccgtgtacactattgaccaacctgggagaggaaggggtaacatcccaacttttaacggacctttcggacagttggaggaagagagtattgttaacactgtgacaggaaattcttcaaaggaaggtgcctgggtgagagataggcttggccctgctcccgggcaatttttcgagaactctcagtttcctagaggctatgaagacaattactttaaggagatgggattcagcccatctatatccagtgatgaaattgttgacgctgttgtgaaactcgtgacccatattggtccttgtgttctggtgactcactcagcatccggcgttcttgggatgagagtggctacacacgcaaagaatgttaggggaattgtggcctatgaaccagctacctcaatcttccccaagggaaaagttccagagataccacctctcgctgataagaaaagccaaatctttcccccattcgaaatacaggagtcttactttaagaaacttgccaagattccaatccaatttgttttcggagataacatccccaagaatccaaaatcagcatattggttcctggactggtggagagtgacaagatacgcacatagtctcagcctggaggccataaacaaattggggggacaagcttcccttttggatcttcctactgcaggattgagaggtaatacacactttcccttcaccgataggaacaatgttcaggtggcttctctcctgtcagactttctgggtaaacacggtctggatcaaaatgagagcaaactcgccgccgccctggaatga

SEQ ID NO: 6 - DNA, 1110 bp, Artificial Sequence
  ALT-2-02 Nucleotide sequence
atggagactgataacgtggagctggcgcagagcaagaggaaggtggttctggctgagcaggggtcgttctacattggggggcggactgtgaccgggcccggcaagttcgacccatcgaagcctgtcattccgtactctaacgagggcgctacgttctacatcaaccagatgtacgtgaatttccaggctcccgtccgcccaaggggcctcccactggtgttctggcacggcgggggcctgacaggccatatctgggagtccactccagatggccgcccagggttccagacactcttcgttcaggacaggcacacagtgtacactattgatcagccagggaggggcagggggaacatccctaccttcaatggcccattcgggcagctggaggaggagtccatcgtgaacaccgtcacgggcaattccagcaaggagggggcttgggtcagggaccggctcggcccggccccagggcagttcttcgagaactctcagttcccccggggctacgaggataattacttcaaggagatgggcttctcaccatccatctcgtctgacgagattgtcgatgccgtggtcaagctcgttacccacatcggcccttgcgttctggtgacgcatagcgcttcgggcgtcctcgggatgagggttgctacacatgcgaagaacgttcgcggcatcgtggcttacgagccggccacttccattttccccaagggcaaggtgccagagatcccaccactggccgacaagaagtcacagatcttcccacctttcgagattcaggagtcctacttcaagaagctcgctaagatccccattcagttcgtgttcggcgacaacattcctaagaatccgaagagcgcgtactggttcctggattggtggcgcgtcacgcgctacgcgcactctctctcactggaggctatcaacaagctcgggggccaggcctcgctcctggacctccctaccgctggcctgagggggaacacccatttcccgttcacggatcggaacaatgtccaggttgcgtccctcctgagcgatttcctcggcaagcacgggctggatcagaatgagtctaagctcgctgcggcgctggagtga

SEQ ID NO: 7 - protein, 362 aa, Artificial Sequence
  ALT-3 Amino acid sequence
METDNVELAQSKRKVVLAEQGSFYIGGRTVTGPGKFDPSKPVIPYSNEGATFYINQMYVNFQAPVRPRGLPLVFWHGGGLTGHIWESTPDGRPGFQTLFVQDRHTVYTIDQPGRGRGNIPTFNGPFGQLEEESIVNTVTGNSSKEGAWVRDRLGPAPGQFFENSQFPRGYEDNYFKEMGFSPSISSDEIVDAVVKLVTHIGPCVLVTHSASGVLGMRVATHAKNVRGIVAYEPATSIFPKGKVPEIPPLADKKSQIFPPFEIQESYFKKLAKIPIQFVFGDNIPKNPKSAYWFLDWWRVTRYAHSLSLEAINKLGGQASLLDLPTAGLRGNTHFPFTDRNNVQVASLLSDFLGKHGLDQNES

SEQ ID NO: 8 - DNA, 1089 bp, Artificial Sequence
  ALT-3-01 Nucleotide sequence
atggagactgataacgtggaactcgcccaatctaagagaaaggtggtgctggctgaacaagggtcattttacatagggggtaggactgttactggtcctggcaagtttgatccatccaaacctgtgataccctacagtaacgaaggagcaacattctatattaaccaaatgtatgttaacttccaggccccagtgagacctaggggacttccattggttttctggcatggaggtggcttgactggtcacatctgggagtctacacctgacggcagacccgggtttcaaaccctcttcgttcaggataggcataccgtgtacactattgaccaacctgggagaggaaggggtaacatcccaacttttaacggacctttcggacagttggaggaagagagtattgttaacactgtgacaggaaattcttcaaaggaaggtgcctgggtgagagataggcttggccctgctcccgggcaatttttcgagaactctcagtttcctagaggctatgaagacaattactttaaggagatgggattcagcccatctatatccagtgatgaaattgttgacgctgttgtgaaactcgtgacccatattggtccttgtgttctggtgactcactcagcatccggcgttcttgggatgagagtggctacacacgcaaagaatgttaggggaattgtggcctatgaaccagctacctcaatcttccccaagggaaaagttccagagataccacctctcgctgataagaaaagccaaatctttcccccattcgaaatacaggagtcttactttaagaaacttgccaagattccaatccaatttgttttcggagataacatccccaagaatccaaaatcagcatattggttcctggactggtggagagtgacaagatacgcacatagtctcagcctggaggccataaacaaattggggggacaagcttcccttttggatcttcctactgcaggattgagaggtaatacacactttcccttcaccgataggaacaatgttcaggtggcttctctcctgtcagactttctgggtaaacacggtctggatcaaaatgagagctga

SEQ ID NO: 9 - DNA, 1089 bp, Artificial Sequence
  ALT-3-02 Nucleotide sequence
atggagactgataacgtggagctggcgcagagcaagaggaaggtggttctggctgagcaggggtcgttctacattggggggcggactgtgaccgggcccggcaagttcgacccatcgaagcctgtcattccgtactctaacgagggcgctacgttctacatcaaccagatgtacgtgaatttccaggctcccgtccgcccaaggggcctcccactggtgttctggcacggcgggggcctgacaggccatatctgggagtccactccagatggccgcccagggttccagacactcttcgttcaggacaggcacacagtgtacactattgatcagccagggaggggcagggggaacatccctaccttcaatggcccattcgggcagctggaggaggagtccatcgtgaacaccgtcacgggcaattccagcaaggagggggcttgggtcagggaccggctcggcccggccccagggcagttcttcgagaactctcagttcccccggggctacgaggataattacttcaaggagatgggcttctcaccatccatctcgtctgacgagattgtcgatgccgtggtcaagctcgttacccacatcggcccttgcgttctggtgacgcatagcgcttcgggcgtcctcgggatgagggttgctacacatgcgaagaacgttcgcggcatcgtggcttacgagccggccacttccattttccccaagggcaaggtgccagagatcccaccactggccgacaagaagtcacagatcttcccacctttcgagattcaggagtcctacttcaagaagctcgctaagatccccattcagttcgtgttcggcgacaacattcctaagaatccgaagagcgcgtactggttcctggattggtggcgcgtcacgcgctacgcgcactctctctcactggaggctatcaacaagctcgggggccaggcctcgctcctggacctccctaccgctggcctgagggggaacacccatttcccgttcacggatcggaacaatgtccaggttgcgtccctcctgagcgatttcctcggcaagcacgggctggatcagaatgagtcttga

SEQ ID NO: 10 - protein, 455 aa, Artificial Sequence
  Amino acid sequence of Herbicide-tolerant Protein
MLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 11 - DNA, 1368 bp, Artificial Sequence
  EPSPS Nucleotide sequence
atgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctga

SEQ ID NO: 12 - DNA, 1322 bp, Arabidopsis thaliana
gtcgacctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 13 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 14 - DNA, 530 bp, Cauliflower mosaic virus
ccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaca

SEQ ID NO: 15 - DNA, 552 bp, Streptomyces viridochromogenes
atgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctga

SEQ ID NO: 16 - DNA, 195 bp, Cauliflower mosaic virus
ctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgg

SEQ ID NO: 17 - DNA, 228 bp, Arabidopsis thaliana
atggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgc

SEQ ID NO: 18 - DNA, 1534 bp, Brassica napus
gattatgacattgctcgtggaatgggacagttatggtatttttttgtaataaattgtttccattgtcatgagattttgaggttaatctatgagacattgaatcacttagcattagggattaagtagtcacaaatcgcattcaagaagctgaagaacacgttatggtctaatggttgtgtctctttattagaaaatgttggtcagtagctatatgcactgtttctgtaaaaccatgttggtgttgtgtttatttcaagacacatgttgagtccgttgattcagagcttttgtcttcgaacacaatctagagagcaaatttgggttcaatttggatatcaatatgggttcgattcagatagaacaataccctttgatgtcgggtttcgatttggttgagattcatttttatcgggtttggttcgattttcgaattcggtttattcgccccctcatagcatctacattctgcagattaatgtacaagttatggaaaaaaaaatgtggttttcgaattcggtttagtagctaaacgttgcttgcagtgtagttatgggaattatgaaacacgaccgaaggtatcaattagaagaacgggtcaacgggtaagtattgagaaattaccggagggtaaaaataaacagtattctttttttttcttaacgaccgaccaaggttaaaaaaagaaaggaggacgagatacaggggcatgactgtaattgtacataagatctgatctttaaaccctaggtttccttcgcatcagcaactataaataattctgagtgccactcttcttcattcctagatctttcgccttatcgctttagctgaggtaagcctttctatacgcatagacgctctcttttctcttctctcgatcttcgttgaaacggtcctcgatacgcataggatcggttagaatcgttaatctatcgtcttagatcttcttgattgttgaattgagcttctaggatgtattgtatcatgtgatggatagttgattggatctctttgagtgaactagctagctttcgatgcgtgtgatttcagtataacaggatccgatgaattatagctcgcttacaattaatctctgcagatttattgtttaatcttggatttgatgctcgttgttgatagaggatcgtttatagaacttattgattctggaattgagcttgtgtgatgtattgtatcatgtgatcgatagctgatggatctatttgagtgaactagcgtacgatcttaagatgagtgtgtattgtgaactgatgattcgagatcagcaaaacaagatctgatgatatcttcgtcttgtatgcatcttgaatttcatgattttttattaattatagctcgcttagctcaaaggatagagcaccacaaaattttattgtggtagaaatcggttcgattccgatagcagcttactgtgatgaatgattttgagatttggtatttgatatatgtctactgtgttgaatgatcgtttatgcattgtttaatcgctgcagatttgcattgacaagtagcc

SEQ ID NO: 19 - DNA, 643 bp, Pisum sativum
agctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaat

SEQ ID NO: 20 - DNA, 25 bp, Artificial Sequence
  Primer 1
ctggaaggcgaggacgtcatcaata

SEQ ID NO: 21 - DNA, 22 bp, Artificial Sequence
  Primer 2
tggcggcattgccgaaatcgag

SEQ ID NO: 22 - DNA, 28 bp, Artificial Sequence
  Probe 1
atgcaggcgatgggcgcccgcatccgta

SEQ ID NO: 23 - DNA, 1992 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 24 - DNA, 1176 bp, Escherichia coli
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 25 - DNA, 29 bp, Artificial Sequence
  Primer 3
gctgtaagagcttactgaaaaaattaaca

SEQ ID NO: 26 - DNA, 18 bp, Artificial Sequence
  Primer 4
cgatctgcaggtcgacgg

SEQ ID NO: 27 - DNA, 27 bp, Artificial Sequence
  Probe 2
tctcttgctaagctgggagctcgatcc